(19) United States
(12) Patent Application Publication
(10) Pub. No.: US 2010/0203650 A1
(43) Pub. Date: Aug. 12, 2010

(54) BIOMOLECULE BINDING LIGANDS

(75) Inventors: Christopher Robin Lowe, Cambridge Cambridgeshire (GB); Abid Hussain, Cambridge Cambridgeshire (GB); Michael Luis Mimmack, Cambridge Cambridgeshire (GB); Jonathan Michael Haigh, Cambridge Cambridgeshire (GB)

Correspondence Address: LEYDIG VOIT & MAYER, LTD, 700 THIRTEENTH ST. NW, SUITE 300, WASHINGTON, DC, 20005-3960, US

(73) Assignee: CAMBRIDGE ENTERPRISE LIMITED, Cambridge Cambridgeshire (GB)

(21) Appl. No.: 12/666,322

(22) PCT Filed: Jun. 27, 2008
(86) PCT No.: PCT/GB2008/002222

§ 371 Date: Dec. 23, 2009

(30) Foreign Application Priority Data
Jul. 6, 2007 (GB) 0713187.3

(51) Int. Cl. G01N 33/68; C40B 40/04; C07C 233/00; C07C 235/02; C07D 209/10; C07D 213/56; C07D 213/55; C07D 473/30; C07C 69/76; C40B 30/00; G01N 33/00; G01N 33/53; C40B 50/00

(52) U.S. Cl. 436/501; 506/15; 564/158; 564/153; 548/507; 546/336; 546/335; 544/276; 560/61; 506/7; 436/96; 436/98; 436/106; 436/128; 436/94; 436/86; 506/23

(57) ABSTRACT

The invention provides biomolecule binding ligands, collections of biomolecule binding ligands, and their use in the purification of biological mixtures and in the identification of ligands having an affinity for a substance. The ligand is a compound of formula (III) or a compound of formula (IV): wherein for compounds of formula (I) one of R^{1a}, R^{1b}, R², R³ and R⁴ is a group comprising a linker attached to a support, and the others of R^{1a}, R^{1b}, R², R³ and R⁴ are independently selected from optionally substituted C₁₋₂₀ alkyl, optionally substituted C₃₋₂₀ heterocyclyl or optionally substituted C₅₋₂₀ aryl, and R^{1a}, R^{1b} and R² are additionally selected from hydrogen, and R² is additionally further selected from —S(═O)R⁵ and —C(═S)NR⁶R⁷, wherein R⁵, R⁶ and R⁷ are independently optionally substituted C₁₋₂₀ alkyl, optionally substituted C₃₋₂₀ heterocyclyl or optionally substituted C₅₋₂₀ aryl, or, optionally, two or more of the others of R^{1a}, R^{1b}, R², R³ and R⁴, together with the atoms to which they are bound, may form a ring; and for compounds of formula (II) one of R^{1a}, R^{1b}, R³ and R⁴ is a group comprising a linker attached to a support, and the others of R^{1a}, R^{1b}, R³ and R⁴ are independently selected optionally substituted C₁₋₂₀ alkyl, optionally substituted C₃₋₂₀ heterocyclyl or optionally substituted C₅₋₂₀ aryl, and R₁ₐ, and R_{1b} are additionally selected from hydrogen, or, optionally, two or more of the others of R^{1a}, R^{1b}, R³ and R⁴, together with the atoms to which they are bound, may form a ring.

### RELATED APPLICATION

This application is related to GB patent application 0713187.3 filed 6 Jul. 2007; the contents of which are incorporated herein by reference in their entirety.

### FIELD OF THE INVENTION

The present invention relates to biomolecule binding ligands, and their use in the purification of biological mixtures. The present invention also relates to collections of ligands, and their use in the identification of compounds having an affinity for a biological molecule.

### BACKGROUND

The modern investigation of human disease may initially commence with an entry-level genome investigation performed by high throughput technologies such as genomic or cDNA microarray studies in parallel. This often results in the identification of mutated gene products or altered patterns of individual gene expression strongly correlated with the monitored disease state and allowing for an extensive candidate gene list to be quickly generated. These DNA/RNA-based studies are themselves limited since they do not take into account the complex interplay of signalling states that proteins can display such as phosphorylation and altered conformation via multiple protein-protein interactions. Therefore the main aim of clinical proteomic studies is often the complete characterisation of numerous candidate proteins strongly implicated in a particular diseases state whether they have been identified by gene expression or direct protein-profiling studies.

This daunting challenge usually requires that a number of individual proteins be purified to homogeneity—a time-consuming and expensive process. This is often required by the scientist to determine various important parameters such as the 3D crystallographic structure, post-translational modification, complex formation with other proteins and production of specific antibodies to aid in tissue localisation studies. It is also important to purify target proteins in order to develop in vitro assay systems that can identify the degree of modulation of biological activity that small-molecule effectors can exert upon the isolated molecule for drug discovery purposes.

This has led to a general increase in the number of important immunotherapeutic proteins that are required for study but has also impacted strongly on the development cost of bringing new biotherapeutic drugs to market in a relatively short period of time. The final product should also possess a fixed level of purity, efficacy, potency, stability as well as clearly defined pharmacokinetic, pharmacodynamic and immunogenic properties. Therefore a series of heavy constraints have been placed on the development of modern purification processes that take into account the speed of introduction, simplicity of operation and economic cost return. Affinity chromatography is still the only recognised technique that can unite the key issues of specific molecular target recognition and suitability for large-scale production processes and thus provides an ‘ideal’ technology to address the rising costs associated with defining a ‘well-characterised biologic’. As much as 50-80% of the total cost of manufacturing a therapeutic product is incurred during downstream processing, purification and polishing and thus many conventional purification protocols are now being substituted with highly selective and sophisticated strategies based on affinity chromatography (Lowe, 2001). The nature of the early development cost for designing and testing new affinity absorbents is still generally considered small as compared to the final savings that can be achieved in the latter large-scale industrial production phase.

The use of conventional affinity ligands such as peptides, oligonucleotides and antibodies (i.e. immunoaffinity purification) have begun to be replaced by second generation, fully-synthetic affinity absorbents derived largely from small-molecule screening programs, modelling studies and fragment-scanning in situ methodologies due to the advent of high throughput combinatorial chemistry techniques and in silico approaches. This has also been supported by the rapid increase in structural information generated by high-quality crystallographic data for many novel target proteins. Biological ligands also suffer from a range of limitations that may include an initial purification cost, lot-to-lot variability, instability and high large-scale production costs. Another important consideration is the ability to effectively clean and reuse an affinity absorbent many times thus extending its lifespan whilst maintaining high activity thereby reducing long-term purification costs. The development of diverse small-molecule combinatorial libraries of affinity ligands displaying large numbers of highly-specific molecular recognition profiles is still an important aim of the protein purification scientist hoping to deliver to industry the latest purified protein with sufficient yield and purity for a cost-effective economic return.

The effective purification of a single protein can rapidly facilitate the production of a novel mAb that recognises this target with native high affinity. At present, 18 therapeutic human mAbs are on the market whereas over 100 mAbs are currently undergoing final clinical trials. So far, five Fab molecules have been approved by the FDA for human use and a single humanised Fab (ranibizumab rhFab) is likely to be approved in the near future.

Such emerging trends in biotherapeutic drug development, and their imminent need for rapid efficient purification, has promoted the development of a novel generic affinity scaffold for ligand design and synthesis. The scaffold of any affinity ligand must comprise the dual capabilities of immobilisation to a solid, insoluble support matrix together with a capacity for complex derivatisation in order to achieve a specific set of molecular interactions and binding constants. This is an absolute requirement necessary to identify and further optimise the separate processes of chromatographic adsorption and desorbtion. We herein report a novel scaffold chemistry for the development of completely synthetic affinity chromatography ligands which can be applied to the purification of immunopharmaceutical targets and other important biomolecules.

Within the field of affinity chromatography there is a continuing need for the provision of new affinity ligands to overcome issues such as poor binding and poor selectivity, and the like, for a substance of interest. Robust methods for producing compounds for use as affinity ligands are also desirable.

Within many fields of biology and chemistry there is a need for the provision of new methods for the identification of compounds capable of acting as ligands for a substance of interest, such as a nucleic acid or peptide. For the identification of new ligand molecules it is considered desirable for the method to be, amongst others: amenable to automation, high throughput, reproducible, and amenable to large scale. Furthermore, it is also desirable to have a method that is capable of exploring a wide and diverse chemical structure space, thereby maximising the likelihood of identifying a ligand having a high affinity for the substance.

### DISCLOSURE OF THE INVENTION

The present invention relates to compounds for use as affinity ligands for the purification of a substance from a mixture. The present invention also relates to the use of compounds and collections of compounds for the identification of ligands having an affinity for a substance.

Accordingly, in the first aspect of the invention there is provided a collection of compounds wherein each member of the collection is independently a compound according to formula (I) or formula (II):

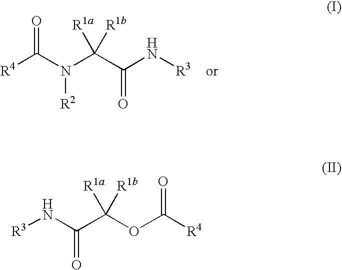

wherein the collection comprises compounds of formula (I) only, compounds of formula (II) only, or a mixture of compounds of formula (I) and (II), and
for compounds of formula (I)
one of R^{1a}, R^{1b}, R², R³ and R⁴ is a group comprising a linker attached to a support,
and the others of R^{1a}, R^{1b}, R², R³ and R⁴ are independently selected from optionally substituted C₁₋₂₀ alkyl, optionally substituted C₃₋₂₀ heterocyclyl or optionally substituted C₅₋₂₀ aryl, and R^{1a}, R^{1b} and R² are additionally selected from hydrogen, and R² is additionally further selected from —S(═O)R⁵ and —C(═S)NR⁶R⁷, wherein R⁵, R⁶ and R⁷ are independently optionally substituted C₁₋₂₀ alkyl, optionally substituted C₃₋₂₀ heterocyclyl or optionally substituted C₅₋₂₀ aryl,
or, optionally, two or more of the others of R^{1a}, R^{1b}, R³ and R⁴, together with the atoms to which they are bound, may form a ring; and
   for compounds of formula (II)
one of R^{1a}, R^{1b}, R³ and R⁴ is a group comprising a linker attached to a support,
and the others of R^{1a}, R^{1b}, R³ and R⁴ are independently selected optionally substituted C₁₋₂₀ alkyl, optionally substituted C₃₋₂₀ heterocyclyl or optionally substituted C₅₋₂₀ aryl, and R^{1a}, and R^{1b} are additionally selected from hydrogen,
or, optionally, two or more of the others of R^{1a}, R^{1b}, R³ and R⁴, together with the atoms to which they are bound, may form a ring.

In a second aspect of the present invention there is provided the use of a collection according to the first aspect of the invention in a process for the identification of a immobilised ligand having affinity for a substance. The process comprises the steps of:
obtaining a collection of compounds according to the first aspect of the invention;
contacting each member of the collection with a mixture comprising a substance; and
analysing the collection to determine to what extent the substance is associated with each collection member.

Preferably, the substance is a nucleic acid or a peptide. The method may include the further step of separating the collection from the mixture.

In a third aspect of the present invention there is provided the use of a collection according to the first aspect of the invention in a process for the generation of a compound having affinity for a substance. The process comprises the steps of:
obtaining a collection of compounds according to the first aspect of the invention;
contacting each member of the collection with a mixture comprising a substance;
analysing the collection to determine to what extent the substance is associated with each collection member;
identifying a library member having an affinity for the substance; and
preparing a compound having a structure based on the collection member.

Preferably, the substance is a nucleic acid or a peptide. The method may include the further step of separating the collection from the mixture.

The compound having affinity for a substance may be prepared by cleaving the linker of a collection that is determined to be associated with the substance. Alternatively, the compound may be prepared by a method comprising the steps of contacting components A, B, C and D together, wherein
A is R^{1a}COR^{1b};
B is R²—NH₂;
C is R³—NC;
D is R⁴—COOH; and
R^{1a}, R^{1b}, R², R³ and R⁴ are independently selected from optionally substituted C₁₋₂₀ alkyl, optionally substituted C₃₋₂₀ heterocyclyl or optionally substituted C₅₋₂₀ aryl, and R^{1a}, R^{1b} and R² are additionally selected from hydrogen, and R² is additionally further selected from —S(═O)R⁵ and —C(═S)NR⁶R⁷, wherein R⁵, R⁶ and R⁷ are independently optionally substituted C₁₋₂₀ alkyl, optionally substituted C₃₋₂₀ heterocyclyl or optionally substituted C₅₋₂₀ aryl,
or, optionally, two or more of R^{1a}, R^{1b}, R², R³ and R⁴ are connected; or the method comprises the step of contacting components A, C and D together, wherein
A is R^{1a}COR^{1b};
C is R³—NC;
D is R⁴—COOH; and
R^{1a}, R^{1b}, R³ and R⁴ are independently selected optionally substituted C₁₋₂₀ alkyl, optionally substituted C₃₋₂₀ heterocyclyl or optionally substituted C₅₋₂₀ aryl, and R^{1a} and R^{1b} are additionally selected from hydrogen,
or, optionally, two or more of the others of R^{1a}, R^{1b}, R³ and R⁴ are connected.

In this latter method step, it is preferred that one component is a structural or functional analogue of the linker.

In a fourth aspect of the invention there is provided a compound of formula (III) or a compound of formula (IV):

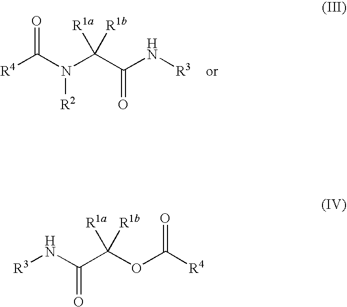

wherein for compounds of formula (III)
one of R^{1a}, R^{1b}, R², R³ and R⁴ is a group comprising a linker attached to a support,
and the others of R^{1a}, R^{1b}, R², R³ and R⁴ are independently selected from optionally substituted C₁₋₂₀ alkyl, optionally substituted C₃₋₂₀ heterocyclyl or optionally substituted C₅₋₂₀ aryl, and R^{1a}, R^{1b} and R² are additionally selected from hydrogen, and R² is additionally further selected from —S(═O)R⁵ and —C(═S)NR⁶R⁷, wherein R⁵, R⁶ and R⁷ are independently optionally substituted C₁₋₂₀ alkyl, optionally substituted C₃₋₂₀ heterocyclyl or optionally substituted C₅₋₂₀ aryl,
or, optionally, two or more of the others of R^{1a}, R^{1b}, R³ and R⁴, together with the atoms to which they are bound, may form a ring; and
   for compounds of formula (IV)
one of R^{1a}, R^{1b}, R³ and R⁴ is a group comprising a linker attached to a support,
and the others of R^{1a}, R^{1b}, R³ and R⁴ are independently selected optionally substituted C₁₋₂₀ alkyl, optionally substituted C₃₋₂₀ heterocyclyl or optionally substituted C₅₋₂₀ aryl, and R^{1a} and R^{1b} are additionally selected from hydrogen,
or, optionally, two or more of the others of R^{1a}, R^{1b}, R³ and R⁴, together with the atoms to which they are bound, may form a ring.

In a fifth aspect of the invention there is provided a separation apparatus for separating a substance from a mixture, wherein the device comprises a compound according to the fourth aspect of the invention.

In a sixth aspect of the present invention there is provided the use of a compound of the fourth aspect of the invention or the use of a separation apparatus of the fifth aspect of the invention in a method for separating a substance from a mixture. The method comprises the steps of:
contacting a mixture comprising a substance with a compound according to the fourth aspect of the invention or a separation apparatus according to the fifth aspect of the invention; and
separating the resulting substance-depleted mixture from the substance immobilised to the compound or device.

In a seventh aspect of the invention, there is provided the use of a compound of the fourth aspect of the invention or the use of a separation apparatus of the fifth aspect of the invention in a method of diagnosis. The method comprises the step of screening a biological sample against a compound with affinity for a substance that is implicated in a particular disease state. The method comprises the steps of:
contacting a biological sample with a compound according to the fourth aspect of the invention or a separation device according to the fifth aspect of the invention; and
analysing the compound or device to what extent the substance that is implicated in a particular disease state is associated with the compound or device.

In an eighth aspect of the invention, there is provided the use of a compound of the fourth aspect of the invention or the use of a separation apparatus of the fifth aspect of the invention in an analytical method for determining the presence of a substance in an analytical sample. The method comprises the step of screening an analytical sample against a compound with affinity for a substance. The method comprises the steps of:
contacting an analytical sample with a compound according to the fourth aspect of the invention or a separation apparatus according to the fifth aspect of the invention; and
analysing the compound or device to determine to what extent the substance is associated with the compound or device.

The present invention also provides in a ninth aspect a method for the preparation of a collection according to the first aspect of the invention. The method comprises the step of contacting components A, B, C and D together, wherein
A is R^{1a}COR^{1b};
B is R²—NH₂;
C is R³—NC;
D is R⁴—COOH; and
one of R^{1a}, R^{1b}, R², R³ and R⁴ is a group comprising a linker attached to a support,
R², R³ and R⁴ are independently selected from optionally substituted C₁₋₂₀ alkyl, optionally substituted C₃₋₂₀ heterocyclyl or optionally substituted C₅₋₂₀ aryl, and R^{1a}, R^{1b} and R² are additionally selected from hydrogen, and R² is additionally further selected from —S(═O)R⁵ and —C(═S)NR⁶R⁷, wherein R⁵, R⁶ and R⁷ are independently optionally substituted C₁₋₂₀ alkyl, optionally substituted C₃₋₂₀ heterocyclyl or optionally substituted C₅₋₂₀ aryl,
or, optionally, two or more of the others of R^{1a}, R^{1b}, R², R³ and R⁴ are connected, wherein the step is repeated one or more times, and for each repeat, one or more of A, B, C or D is varied;
or the method comprises the step of contacting components A, C and D together, wherein
A is R^{1a}COR^{1b};
C is R³—NC;
D is R⁴—COOH; and
one of R^{1a}, R^{1b}, R³ and R⁴ is a group comprising a linker attached to a support, and the others of R^{1a}, R^{1b}, R³ and R⁴ are independently selected optionally substituted C₁₋₂₀ alkyl, optionally substituted C₃₋₂₀ heterocyclyl or optionally substituted C₅₋₂₀ aryl, and R^{1a} and R^{1b} are additionally selected from hydrogen,
or, optionally, two or more of the others of R^{1a}, R^{1b}, R³ and R⁴ are connected,
wherein the step is repeated one or more times, and for each repeat, one or more of A, C or D is varied.

Preferably the steps are performed at the same time. Preferably each step is performed in a discrete reaction pot.

The present invention also provides in a tenth aspect a method for the preparation of a compound according to the fourth aspect of the invention. The method comprises the step of contacting components A, B, C and D together, wherein
A is R^{1a}COR^{1b};
B is R²—NH₂;
C is R³—NC;
D is R⁴—COOH; and
one of R^{1a}, R^{1b}, R³ and R⁴ is a group comprising a linker attached to a support,
and the others R^{1a}, R^{1b}, R², R³ and R⁴ are independently selected from optionally substituted C₁₋₂₀ alkyl, optionally substituted C₃₋₂₀ heterocyclyl or optionally substituted C₅₋₂₀ aryl, and R^{1a}, R^{1b} and R² are additionally selected from hydrogen, and R² is additionally further selected from —S(═O)R⁵ and —C(═S)NR⁶R⁷, wherein R⁵, R⁶ and R⁷ are independently optionally substituted C₁₋₂₀ alkyl, optionally substituted C₃₋₂₀ heterocyclyl or optionally substituted C₅₋₂₀ aryl,
or, optionally, two or more of the others of R^{1a}, R^{1b}, R², R³ and R⁴ are connected;
or the method comprises the step of contacting components A, C and D together, wherein
A is R^{1a}COR^{1b};
C is R³—NC;
D is R⁴—COOH; and
one of R^{1a}, R^{1b}, R³ and R⁴ is a group comprising a linker attached to a support,
and the others of R^{1a}, R^{1b}, R³ and R⁴ are independently selected optionally substituted C₁₋₂₀ alkyl, optionally substituted C₃₋₂₀ heterocyclyl or optionally substituted C₅₋₂₀ aryl, and R^{1a} and R^{1b} are additionally selected from hydrogen,
or, optionally, two or more of the others of R^{1a}, R^{1b}, R³ and R⁴ are connected.

In another aspect of the invention, there is provided a collection of compounds obtainable by the method of the ninth aspect of the invention.

### BRIEF DESCRIPTION OF THE FIGURES

FIG. 1 shows the (A) ¹H NMR and (B) ¹³C NMR spectra for compound 5.

FIG. 2 shows the fluorescence images of the ligands used for qualitative evidence of in situ Ugi scaffold formation.

FIG. 3 shows the results of an assay in which an Ugi reaction-produced library was screened for hIgG binding (μgml⁻¹) based on non-optimised standard chromatographic conditions (c.v.: 200 μl; hIgG load: 500 μgml⁻¹ (1 c.v.); ligand density: 24 μmol g⁻¹ moist weight gel). The labels A1-8 and C1-8 identify the amine and carboxylic components used in the Ugi reaction as described in detail in the experimental section.

FIG. 4 shows the results of an assay in which the library described in relation to FIG. 3 was screened for hFab binding.

FIG. 5 shows the results of an assay in which the library described in relation to FIG. 3 was screened for hFc binding.

FIG. 6 shows a comparison of % binding and elution for hIgG lead candidate ligands. Non-optimised binding (10 mM Na₂HPO₄, 150 mM NaCl, pH 7.4)) and elution (0.1M NaHCO₃, 10% (v/v) ethylene glycol, pH 10.0) conditions were applied. % elution is represented as a percentage of bound protein. (Ligand density: 17.5 μmol g⁻¹ moist weight gel)

FIG. 7 shows a comparison of % binding and elution for hFab lead ligands under the conditions described in relation to FIG. 6.

FIG. 8 shows a comparison of % binding and elution for hFc lead ligands under the conditions described in relation to FIG. 6.

FIG. 9 shows the results of a Factor VIII binding study using columns packed with selected ligand compounds 4U, 8U and 9U compared to the triazine ligand 34/43.

FIG. 10 shows the elution behaviour of selected ligand compounds 4U, 8U and 9U compared to the triazine ligand 34/43.

FIG. 11 shows Factor VIII microplate assay results of selected ligand compounds 4U, 6U, 7U, 8U, 9U, 10U, 11U, 12U, 13U and 14U compared to the triazine ligand 34/43.

FIG. 12 shows differential binding modes identified for selected ligand compounds 4U, 9U and 14U.

FIG. 13 shows differential binding modes identified for selected ligand compounds 4U, 16U, 17U and 14U.

FIG. 14 shows the results of a Factor VIII elution from selected ligand compounds 8U, 14U, 16U and 17U.

### DETAILED DESCRIPTION OF THE INVENTION

### Definitions

### R^{1a}, R^{1b}, R², R³, R⁴, R⁵, R⁶ and R⁷

C₁₋₂₀ Alkyl: The term “alkyl” as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated or unsaturated (e.g. partially unsaturated, fully unsaturated). Thus, the term “alkyl” includes the sub-classes alkenyl, alkynyl, cycloalkyl, cycloalkyenyl, cylcoalkynyl, etc., discussed below.

In the context of alkyl groups, the prefixes (e.g. C₁₋₄, C₁₋₇, C₁₋₂₀, C₂₋₇, C₃₋₇, etc.) denote the number of carbon atoms, or range of number of carbon atoms. For example, the term “C₁₋₄ alkyl”, as used herein, pertains to an alkyl group having from 1 to 4 carbon atoms. Examples of groups of alkyl groups include C₁₋₄ alkyl (“lower alkyl”), C₁₋₇ alkyl, and C₁₋₂₀ alkyl. Note that the first prefix may vary according to other limitations; for example, for unsaturated alkyl groups, the first prefix must be at least 2; for cyclic alkyl groups, the first prefix must be at least 3; etc.

Examples of (unsubstituted) saturated alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉), decyl (C₁₀), undecyl (C₁₁), dodecyl (C₁₂), tridecyl (C₁₃), tetradecyl (C₁₄), pentadecyl (C₁₅), and eicodecyl (C₂₀).

Examples of (unsubstituted) saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of (unsubstituted) saturated branched alkyl groups include, but are not limited to, iso-propyl (C₃), iso-butyl (C₄), sec-butyl (C₄), tert-butyl (C₄), iso-pentyl (C₅), and neo-pentyl (C₅).

Alkenyl: The term “alkenyl”, as used herein, pertains to an alkyl group having one or more carbon-carbon double bonds. Examples of alkenyl groups include C₂₋₄ alkenyl, C₂₋₇ alkenyl, C₂₋₂₀ alkenyl.

Examples of (unsubstituted) unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

Alkynyl: The term “alkynyl”, as used herein, pertains to an alkyl group having one or more carbon-carbon triple bonds. Examples of alkynyl groups include C₂₋₄ alkynyl, C₂₋₇ alkynyl, C₂₋₂₀ alkynyl.

Examples of (unsubstituted) unsaturated alkynyl groups include, but are not limited to, ethynyl (ethinyl, —C≡CH) and 2-propynyl (propargyl, —CH₂—C≡CH).

Cycloalkyl: The term “cycloalkyl”, as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g. partially unsaturated, fully unsaturated), which moiety has from 3 to 20 carbon atoms (unless otherwise specified), including from 3 to 20 ring atoms. Thus, the term “cycloalkyl” includes the sub-classes cycloalkenyl and cycloalkynyl. Preferably, each ring has from 3 to 7 ring atoms. Examples of groups of cycloalkyl groups include C₃₋₂₀ cycloalkyl, C₃₋₁₅ cycloalkyl, C₃₋₁₀ cycloalkyl, C₃₋₇ cycloalkyl.

Examples of cycloalkyl groups include, but are not limited to, those derived from:

Saturated Monocyclic Hydrocarbon Compounds:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀);

Unsaturated Monocyclic Hydrocarbon Compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₅);

Saturated Polycyclic Hydrocarbon Compounds:

thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀);

Unsaturated Polycyclic Hydrocarbon Compounds:

camphene (C₁₀), limonene (C₁₀), pinene (C₁₀);

Polycyclic Hydrocarbon Compounds Having an Aromatic Ring:

indene (C₉), indane (e.g., 2,3-dihydro-1H-indene) (C₉), tetraline (1,2,3,4-tetrahydronaphthalene) (C₁₀), acenaphthene (C₁₂), fluorene (C₁₃), phenalene (C₁₃), acephenanthrene (C₁₅), aceanthrene (C₁₆), cholanthrene (C₂₀).

C₃₋₂₀ Heterocyclyl: The term “heterocyclyl”, as used herein, pertains to a monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₂₀ heterocyclyl, C₅₋₂₀ heterocyclyl, C₃₋₁₅ heterocyclyl, C₅₋₁₅ heterocyclyl, C₃₋₁₂ heterocyclyl, C₅₋₁₂ heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₅), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and,
N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Spiro-C₃₋₇ cycloalkyl or heterocyclyl: The term “spiro C₃₋₇ cycloalkyl or heterocyclyl” as used herein, refers to a C₃₋₇ cycloalkyl or C₃₋₇ heterocyclyl ring joined to another ring by a single atom common to both rings.

C₅₋₂₀ Aryl: The term “aryl” as used herein, pertains to a monovalent moiety obtained by removing a hydrogen atom from an aromatic ring atom of an aromatic compound, said compound having one ring, or two or more rings (e.g., fused), and wherein at least one of said ring(s) is an aromatic ring. Preferably, each ring has from 5 to 7 ring atoms. Preferably, the aryl group is a C₅₋₂₀ aryl group.

The ring atoms may be all carbon atoms, as in “carboaryl groups” in which case the group may conveniently be referred to as a “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), and pyrene (C₁₆).

Alternatively, the ring atoms may include one or more heteroatoms, including but not limited to oxygen, nitrogen, and sulfur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, tetrazole and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine) and triazine.

The heteroaryl group may be bonded via a carbon or hetero ring atom.

Examples of C₅₋₂₀ heteroaryl groups which comprise fused rings, include, but are not limited to, C₉ heteroaryl groups derived from benzofuran, isobenzofuran, benzothiophene, indole, isoindole; C₁₀ heteroaryl groups derived from quinoline, isoquinoline, benzodiazine, pyridopyridine; C₁₄ heteroaryl groups derived from acridine and xanthene.

The above alkyl, heterocyclyl and aryl groups, whether alone or part of another substituent, may themselves optionally be substituted with one or more groups selected from themselves and the additional substituents listed below.

Hydrogen: —H. Note that if the substituent at a particular position is hydrogen, it may be convenient to refer to the compound or group as being “unsubstituted” at that position.

Halo: —F, —Cl, —Br, and —I.

Hydroxy: —OH.

Ether: —OR, wherein R is an ether substituent, for example, a C₁₋₇alkyl group (also referred to as a C₁₋₇alkoxy group, discussed below), a C₃₋₂₀heterocyclyl group (also referred to as a C₃₋₂₀heterocyclyloxy group), or a C₅₋₂₀aryl group (also referred to as a C₅₋₂₀aryloxy group), preferably a C₁₋₇alkyl group.

Alkoxy: —OR, wherein R is an alkyl group, for example, a C₁₋₇alkyl group. Examples of C₁₋₇alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Acetal: —CH(OR¹)(OR²), wherein R¹ and R² are independently acetal substituents, for example, a C₁₋₇alkyl group, a C₃₋₂₀heterocyclyl group, or a C₅₋₂₀aryl group, preferably a C₁₋₇alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe)(OEt).

Hemiacetal: —CH(OH)(OR¹), wherein R¹ is a hemiacetal substituent, for example, a C₁₋₇alkyl group, a C₃₋₂₀heterocyclyl group, or a C₅₋₂₀aryl group, preferably a C₁₋₇alkyl group. Examples of hemiacetal groups include, but are not limited to, —CH(OH)(OMe) and —CH(OH)(OEt).

Ketal: —CR(OR¹)(OR²), where R¹ and R² are as defined for acetals, and R is a ketal substituent other than hydrogen, for example, a C₁₋₇alkyl group, a C₃₋₂₀heterocyclyl group, or a C₅₋₂₀aryl group, preferably a C₁₋₇alkyl group. Examples ketal groups include, but are not limited to, —C(Me)(OMe)₂, —C(Me)(OEt)₂, —C(Me)(OMe)(OEt), —C(Et)(OMe)₂, —C(Et)(OEt)₂, and —C(Et)(OMe)(OEt).

Hemiketal: —CR(OH)(OR¹), where R¹ is as defined for hemiacetals, and R is a hemiketal substituent other than hydrogen, for example, a C₁₋₇alkyl group, a C₃₋₂₀heterocyclyl group, or a C₅₋₂₀aryl group, preferably a C₁₋₇alkyl group. Examples of hemiacetal groups include, but are not limited to, —C(Me)(OH)(OMe), —C(Et)(OH)(OMe), —C(Me)(OH)(OEt), and —C(Et)(OH)(OEt).

Oxo (keto, -one): ═O.

Thione (thioketone): ═S.

Imino (imine): ═NR, wherein R is an imino substituent, for example, hydrogen, C₁₋₇alkyl group, a C₃₋₂₀heterocyclyl group, or a C₅₋₂₀aryl group, preferably hydrogen or a C₁₋₇alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇alkyl group (also referred to as C₁₋₇alkylacyl or C₁₋₇alkanoyl), a C₃₋₂₀heterocyclyl group (also referred to as C₃₋₂₀heterocyclylacyl), or a C₅₋₂₀aryl group (also referred to as C₅₋₂₀arylacyl), preferably a C₁₋₇alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Carboxy (carboxylic acid): —C(═O)OH.

Boronic acid: —B(OH)₂.

Boronic acid: —B(OR)₂, where R is alkyl or aryl.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR, wherein R is an ester substituent, for example, a C₁₋₇alkyl group, a C₃₋₂₀heterocyclyl group, or a C₅₋₂₀aryl group, preferably a C₁₋₇alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇alkyl group, a C₃₋₂₀heterocyclyl group, or a C₅₋₂₀aryl group, preferably a C₁₋₇alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₇alkyl group, a C₃₋₂₀heterocyclyl group, or a C₅₋₂₀aryl group, preferably a C₁₋₇alkyl group. Examples of ester groups include, but are not limited to, —OC(═O)OCH₃, —OC(═O)OCH₂CH₃, —OC(═O)OC(CH₃)₃, and —OC(═O)OPh.

Amino: —NR¹R², wherein R¹ and R² are independently amino substituents, for example, hydrogen, a C₁₋₇alkyl group (also referred to as C₁₋₇alkylamino or di-C₁₋₇alkylamino), a C₃₋₂₀heterocyclyl group, or a C₃₋₂₀aryl group, preferably H or a C₁₋₇alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (−⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Acylamido (acylamino): —NR¹C(═O)R², wherein R¹ is an amide substituent, for example, hydrogen, a C₁₋₇alkyl group, a C₃₋₂₀heterocyclyl group, or a C₅₋₂₀aryl group, preferably hydrogen or a C₁₋₇alkyl group, and R² is an acyl substituent, for example, a C₁₋₇alkyl group, a C₃₋₂₀heterocyclyl group, or a C₅₋₂₀aryl group, preferably hydrogen or a C₁₋₇alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

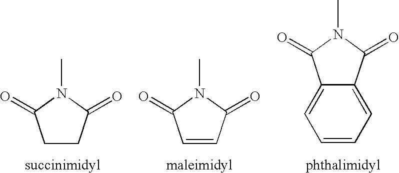

Aminocarbonyloxy: —OC(═O)NR¹R², wherein R¹ and R² are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.

Ureido: —N(R¹)CONR²R³ wherein R² and R³ are independently amino substituents, as defined for amino groups, and R¹ is a ureido substituent, for example, hydrogen, a C₁₋₇alkyl group, a C₃₋₂₀heterocyclyl group, or a C₅₋₂₀aryl group, preferably hydrogen or a C₁₋₇alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

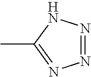

Imino: ═NR, wherein R is an imino substituent, for example, for example, hydrogen, a C₁₋₇alkyl group, a C₃₋₂₀heterocyclyl group, or a C₅₋₂₀aryl group, preferably H or a C₁₋₇alkyl group. Examples of imino groups include, but are not limited to, ═NH, ═NMe, and ═NEt.

Amidine (amidino): —C(═NR)NR₂, wherein each R is an amidine substituent, for example, hydrogen, a C₁₋₇alkyl group, a C₃₋₂₀heterocyclyl group, or a C₅₋₂₀aryl group, preferably H or a C₁₋₇alkyl group. Examples of amidine groups include, but are not limited to, —C(═NH)NH₂, —C(═NH)NMe₂, and —C(═NMe)NMe₂.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇alkyl group (also referred to as a C₁₋₇alkylthio group), a C₃₋₂₀heterocyclyl group, or a C₅₋₂₀aryl group, preferably a C₁₋₇alkyl group. Examples of C₁₋₇alkylthio groups include, but are not limited to, —SCH₃ and —SCH₂CH₃.

Disulfide: —SS—R, wherein R is a disulfide substituent, for example, a C₁₋₇alkyl group, a C₃₋₂₀heterocyclyl group, or a C₅₋₂₀aryl group, preferably a C₁₋₇alkyl group (also referred to herein as C₁₋₇alkyl disulfide). Examples of C₁₋₇alkyl disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇alkyl group, a C₃₋₂₀heterocyclyl group, or a C₅₋₂₀aryl group, preferably a C₁₋₇alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇alkyl group, a C₃₋₂₀heterocyclyl group, or a C₅₋₂₀aryl group, preferably a C₁₋₇alkyl group, including, for example, a fluorinated or perfluorinated C₁₋₇alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇alkyl group, a C₃₋₂₀heterocyclyl group, or a C₅₋₂₀aryl group, preferably a C₁₋₇alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇alkyl group, a C₃₋₂₀heterocyclyl group, or a C₅₋₂₀aryl group, preferably a C₁₋₇alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇alkyl group, a C₃₋₂₀heterocyclyl group, or a C₅₋₂₀aryl group, preferably a C₁₋₇alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇alkyl group, a C₃₋₂₀heterocyclyl group, or a C₅₋₂₀aryl group, preferably a C₁₋₇alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇alkyl group, a C₃₋₂₀heterocyclyl group, or a C₅₋₂₀aryl group, preferably a C₁₋₇alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇alkyl group, a C₃₋₂₀heterocyclyl group, or a C₅₋₂₀aryl group, preferably a C₁₋₇alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substituent, as defined for amino groups, and R is a sulfinamino substituent, for example, a C₁₋₇alkyl group, a C₃₋₂₀heterocyclyl group, or a C₅₋₂₀aryl group, preferably a C₁₋₇alkyl group. Examples of sulfinamino groups include, but are not limited to, —NHS(═O)CH₃ and —N(CH₃)S(═O)C₆H₅.

Phosphino (phosphine): —PR₂, wherein R is a phosphino substituent, for example, —H, a C₁₋₇alkyl group, a C₃₋₂₀heterocyclyl group, or a C₅₋₂₀aryl group, preferably —H, a C₁₋₇alkyl group, or a C₅₋₂₀aryl group. Examples of phosphino groups include, but are not limited to, —PH₂, —P(CH₃)₂, —P(CH₂CH₃)₂, —P(t-Bu)₂, and —P(Ph)₂.

Phospho: —P(—O)₂.

Phosphinyl (phosphine oxide): —P(═O)R₂, wherein R is a phosphinyl substituent, for example, a C₁₋₇alkyl group, a C₃₋₂₀heterocyclyl group, or a C₅₋₂₀aryl group, preferably a C₁₋₇alkyl group or a C₅₋₂₀aryl group. Examples of phosphinyl groups include, but are not limited to, —P(═O)(CH₃)₂, —P(═O)(CH₂CH₃)₂, —P(═O)(t-Bu)₂, and —P(═O)(Ph)₂.

Phosphonic acid (phosphono): —P(═O)(OH)₂.

Phosphonate (phosphono ester): —P(═O)(OR)₂, where R is a phosphonate substituent, for example, —H, a C₁₋₇alkyl group, a C₃₋₂₀heterocyclyl group, or a C₅₋₂₀aryl group, preferably —H, a C₁₋₇alkyl group, or a C₅₋₂₀aryl group. Examples of phosphonate groups include, but are not limited to, —P(═O)(OCH₃)₂, —P(═O)(OCH₂CH₃)₂, —P(═O)(O-t-Bu)₂, and —P(═O)(OPh)₂.

Phosphoric acid (phosphonooxy): —OP(═O)(OH)₂.

Phosphate (phosphonooxy ester): —OP(═O)(OR)₂, where R is a phosphate substituent, for example, —H, a C₁₋₇alkyl group, a C₃₋₂₀heterocyclyl group, or a C₅₋₂₀aryl group, preferably —H, a C₁₋₇alkyl group, or a C₅₋₂₀aryl group. Examples of phosphate groups include, but are not limited to, —OP(═O)(OCH₃)₂, —OP(═O)(OCH₂CH₃)₂, —OP(═O)(O-t-Bu)₂, and —OP(═O)(OPh)₂.

Phosphorous acid: —OP(OH)₂.

Phosphite: —OP(OR)₂, where R is a phosphite substituent, for example, —H, a C₁₋₇alkyl group, a C₃₋₂₀heterocyclyl group, or a C₅₋₂₀aryl group, preferably —H, a C₁₋₇alkyl group, or a C₅₋₂₀aryl group. Examples of phosphite groups include, but are not limited to, —OP(OCH₃)₂, —OP(OCH₂CH₃)₂, —OP(O-t-Bu)₂, and —OP(OPh)₂.

Phosphoramidite: —OP(OR¹)—NR²₂, where R¹ and R² are phosphoramidite substituents, for example, —H, a (optionally substituted) C₁₋₇alkyl group, a C₃₋₂₀heterocyclyl group, or a C₅₋₂₀aryl group, preferably —H, a C₁₋₇alkyl group, or a C₅₋₂₀aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR¹)—NR²₂, where R¹ and R² are phosphoramidate substituents, for example, —H, a (optionally substituted) C₁₋₇alkyl group, a C₃₋₂₀heterocyclyl group, or a C₅₋₂₀aryl group, preferably —H, a C₁₋₇alkyl group, or a C₅₋₂₀aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

Silyl: —SiR₃, where R is a silyl substituent, for example, —H, a C₁₋₇alkyl group, a C₃₋₂₀heterocyclyl group, or a C₅₋₂₀aryl group, preferably —H, a C₁₋₇alkyl group, or a C₅₋₂₀aryl group. Examples of silyl groups include, but are not limited to, —SiH₃, —SiH₂(CH₃), —SiH(CH₃)₂, —Si(CH₃)₃, —Si(Et)₃, —Si(iPr)₃, —Si(tBu)(CH₃)₂, and —Si(tBu)₃.

Oxysilyl: —Si(OR)₃, where R is an oxysilyl substituent, for example, —H, a C₁₋₇alkyl group, a C₃₋₂₀heterocyclyl group, or a C₅₋₂₀aryl group, preferably —H, a C₁₋₇alkyl group, or a C₅₋₂₀aryl group. Examples of oxysilyl groups include, but are not limited to, —Si(OH)₃, —Si(OMe)₃, —Si(OEt)₃, and —Si(OtBu)₃.

Siloxy (silyl ether): —OSiR₃, where SiR₃ is a silyl group, as discussed above.

Oxysiloxy: —OSi(OR)₃, wherein OSi(OR)₃ is an oxysilyl group, as discussed above.

In many cases, substituents are themselves substituted.

For example, a C₁₋₇alkyl group may be substituted with, for example:

hydroxy (also referred to as a hydroxy-C₁₋₇alkyl group);
halo (also referred to as a halo-C₁₋₇alkyl group);
amino (also referred to as a amino-C₁₋₇alkyl group);
carboxy (also referred to as a carboxy-C₁₋₇alkyl group);
C₁₋₇alkoxy (also referred to as a C₁₋₇alkoxy-C₁₋₇alkyl group);
C₅₋₂₀aryl (also referred to as a C₅₋₂₀aryl-C₁₋₇alkyl group).

Similarly, a C₅₋₂₀aryl group may be substituted with, for example:

hydroxy (also referred to as a hydroxy-C₅₋₂₀aryl group);
halo (also referred to as a halo-C₅₋₂₀aryl group);
amino (also referred to as an amino-C₅₋₂₀aryl group, e.g., as in aniline);
carboxy (also referred to as an carboxy-C₅₋₂₀aryl group, e.g., as in benzoic acid);
C₁₋₇alkyl (also referred to as a C₁₋₇alkyl-C₅₋₂₀aryl group, e.g., as in toluene);
C₁₋₇alkoxy (also referred to as a C₁₋₇alkoxy-C₅₋₂₀aryl group, e.g., as in anisole);
C₅₋₂₀aryl (also referred to as a C₅₋₂₀aryl-C₅₋₂₀aryl, e.g., as in biphenyl).

These and other specific examples of such substituted-substituents are described below.

Hydroxy-C₁₋₇alkyl: The term “hydroxy-C₁₋₇alkyl,” as used herein, pertains to a C₁₋₇alkyl group in which at least one hydrogen atom (e.g., 1, 2, 3) has been replaced with a hydroxy group. Examples of such groups include, but are not limited to, —CH₂OH, —CH₂CH₂OH, and —CH(OH)CH₂OH.

Halo-C₁₋₇alkyl group: The term “halo-C₁₋₇alkyl,” as used herein, pertains to a C₁₋₇alkyl group in which at least one hydrogen atom (e.g., 1, 2, 3) has been replaced with a halogen atom (e.g., F, Cl, Br, I). If more than one hydrogen atom has been replaced with a halogen atom, the halogen atoms may independently be the same or different. Every hydrogen atom may be replaced with a halogen atom, in which case the group may conveniently be referred to as a C₁₋₇ perhaloalkyl group.” Examples of such groups include, but are not limited to, —CF₃, —CHF₂, —CH₂F, —CCl₃, —CBr₃, —CH₂CH₂F, —CH₂CHF₂, and —CH₂CF₃.

Amino-C₁₋₇alkyl: The term “amino-C₁₋₇alkyl,” as used herein, pertains to a C₁₋₇alkyl group in which at least one hydrogen atom (e.g., 1, 2, 3) has been replaced with an amino group. Examples of such groups include, but are not limited to, —CH₂NH₂, —CH₂CH₂NH₂, and —CH₂CH₂N(CH₃)₂.

Carboxy-C₁₋₇alkyl: The term “carboxy-C₁₋₇alkyl,” as used herein, pertains to a C₁₋₇alkyl group in which at least one hydrogen atom (e.g., 1, 2, 3) has been replaced with a carboxy group. Examples of such groups include, but are not limited to, —CH₂COOH and —CH₂CH₂COOH.

C₁₋₇alkoxy-C₁₋₇alkyl: The term “C₁₋₇alkoxy-C₁₋₇alkyl,” as used herein, pertains to a C₁₋₇alkyl group in which at least one hydrogen atom (e.g., 1, 2, 3) has been replaced with a C₁₋₇alkoxy group. Examples of such groups include, but are not limited to, —CH₂OCH₃, —CH₂CH₂OCH₃, and, —CH₂CH₂OCH₂CH₃

C₅₋₂₀aryl-C₁₋₇alkyl: The term “C₅₋₂₀aryl-C₁₋₇alkyl,” as used herein, pertains to a C₁₋₇alkyl group in which at least one hydrogen atom (e.g., 1, 2, 3) has been replaced with a C₅₋₂₀aryl group. Examples of such groups include, but are not limited to, benzyl (phenylmethyl, PhCH₂—), benzhydryl (Ph₂CH—), trityl (triphenylmethyl, Ph₃C—), phenethyl (Phenylethyl, Ph-CH₂CH₂—), styryl (Ph-CH═CH—), cinnamyl (Ph-CH═CH—CH₂—).

Hydroxy-C₅₋₂₀aryl: The term “hydroxy-C₅₋₂₀aryl,” as used herein, pertains to a C₅₋₂₀aryl group in which at least one hydrogen atom (e.g., 1, 2, 3) has been substituted with an hydroxy group. Examples of such groups include, but are not limited to, those derived from: phenol, naphthol, pyrocatechol, resorcinol, hydroquinone, pyrogallol, phloroglucinol.

Halo-C₅₋₂₀aryl: The term “halo-C₅₋₂₀aryl,” as used herein, pertains to a C₅₋₂₀aryl group in which at least one hydrogen atom (e.g., 1, 2, 3) has been substituted with a halo (e.g., F, Cl, Br, I) group. Examples of such groups include, but are not limited to, halophenyl (e.g., fluorophenyl, chlorophenyl, bromophenyl, or iodophenyl, whether ortho-, meta-, or para-substituted), dihalophenyl, trihalophenyl, tetrahalophenyl, and pentahalophenyl.

C₁₋₇alkyl-C₅₋₂₀aryl: The term “C₁₋₇alkyl-C₅₋₂₀aryl,” as used herein, pertains to a C₅₋₂₀aryl group in which at least one hydrogen atom (e.g., 1, 2, 3) has been substituted with a C₁₋₇alkyl group. Examples of such groups include, but are not limited to, tolyl (from toluene), xylyl (from xylene), mesityl (from mesitylene), and cumenyl (or cumyl, from cumene), and duryl (from durene).

Hydroxy-C₁₋₇alkoxy: —OR, wherein R is a hydroxy-C₁₋₇alkyl group. Examples of hydroxy-C₁₋₇alkoxy groups include, but are not limited to, —OCH₂OH, —OCH₂CH₂OH, and —OCH₂CH₂CH₂OH.

Halo-C₁₋₇alkoxy: —OR, wherein R is a halo-C₁₋₇alkyl group. Examples of halo-C₁₋₇alkoxy groups include, but are not limited to, —OCF₃, —OCHF₂, —OCH₂F, —OCCl₃, —OCBr₃, —OCH₂CH₂F, —OCH₂CHF₂, and —OCH₂CF₃.

Carboxy-C₁₋₇alkoxy: —OR, wherein R is a carboxy-C₁₋₇alkyl group. Examples of carboxy-C₁₋₇alkoxy groups include, but are not limited to, —OCH₂COOH, —OCH₂CH₂COOH, and —OCH₂CH₂CH₂COOH.

C₁₋₇alkoxy-C₁₋₇alkoxy: —OR, wherein R is a C₁₋₇alkoxy-C₁₋₇alkyl group. Examples of C₁₋₇alkoxy-C₁₋₇alkoxy groups include, but are not limited to, —OCH₂OCH₃, —OCH₂CH₂OCH₃, and —OCH₂CH₂OCH₂CH₃.

C₅₋₂₀aryl-C₁₋₇alkoxy: —OR, wherein R is a C₅₋₂₀aryl-C₁₋₇alkyl group. Examples of such groups include, but are not limited to, benzyloxy, benzhydryloxy, trityloxy, phenethoxy, styryloxy, and cimmamyloxy.

C₁₋₇alkyl-C₅₋₂₀aryloxy: —OR, wherein R is a C₁₋₇alkyl-C₅₋₂₀aryl group. Examples of such groups include, but are not limited to, tolyloxy, xylyloxy, mesityloxy, cumenyloxy, and duryloxy.

Amino-C₁₋₇alkyl-amino: The term “amino-C₁₋₇alkyl-amino,” as used herein, pertains to an amino group, —NR¹R², in which one of the substituents, R¹ or R², is itself a amino-C₁₋₇alkyl group (—C₁₋₇alkyl-NR³R⁴). The amino-C₁₋₇alkylamino group may be represented, for example, by the formula —NR¹—C₁₋₇alkyl-NR³R⁴. Examples of such groups include, but are not limited to, groups of the formula —NR¹(CH₂)ₙNR¹R², where n is 1 to 6 (for example, —NHCH₂NH₂, —NH(CH₂)₂NH₂, —NH(CH₂)₃NH₂, —NH(CH₂)₄NH₂, —NH(CH₂)₅NH₂, —NH(CH₂)₆NH₂), —NHCH₂NH(Me), —NH(CH₂)₂NH(Me), —NH(CH₂)₃NH(Me), —NH(CH₂)₄NH(Me), —NH(CH₂)₅NH(Me), —NH(CH₂)₆NH(Me), —NHCH₂NH(Et), —NH(CH₂)₂NH(Et), —NH(CH₂)₃NH(Et), —NH(CH₂)₄NH(Et), —NH(CH₂)₅NH(Et), and —NH(CH₂)₆NH(Et).

### Bidentate Substituents and Bidentate Reagents

The term “bidentate substituents,” as used herein, pertains to substituents which have two points of covalent attachment, and which act as a linking group between two other moieties.

The term “bidentate reagents,” as used herein, pertains to reagents which have two functional groups that may be used as points of covalent attachment. The bidentate reagent may be used to generate a product having a bidentate substituent.

In some cases (A), a bidentate substituent is covalently bound to a single atom (A¹). In some cases (B), a bidentate substituent is covalently bound to two different atoms (A¹ and A²), and so serves as a linking group therebetween.

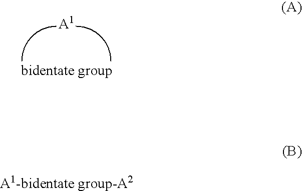

Within (B), in some cases (C), a bidentate substituent is covalently bound to two different atoms, which themselves are not otherwise covalently linked (directly, or via intermediate groups). In some cases (D), a bidentate substituent is covalently bound to two different atoms, which themselves are already covalently linked (directly, or via intermediate groups); in such cases, a cyclic structure results. In some cases, the bidentate group is covalently bound to vicinal atoms, that is, adjacent atoms, in the parent group.

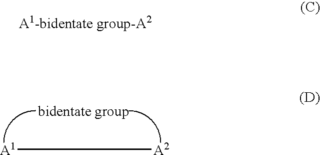

In some cases (A and D), the bidentate group, together with the atom(s) to which it is attached (and any intervening atoms, if present) form an additional cyclic structure. In this way, the bidentate substituent may give rise to a cyclic or polycyclic (e.g., fused, bridged, spiro) structure, which may be aromatic.

Examples of bidentate groups include, but are not limited to, C₁₋₇alkylene groups, C₃₋₂₀ heterocyclylene groups, and C₅₋₂₀arylene groups, and substituted forms thereof.

### Support

The supports described herein may be any structure that allows the compound to be physically separated from a mixture containing a substance. The support may be a solid support or a soluble support.

The solid support may be an insoluble, functionalized, polymeric material to which a compound or reagent may be attached (often via a linker) allowing them to be readily separated (by filtration, centrifugation, etc.) from excess reagents, soluble reaction by-products, or solvents.

The soluble support may be an attachment which renders the compound soluble under conditions for library synthesis, but which can be readily separated from most other soluble components when desired by some simple physical process. This process has been termed liquid-phase chemistry. Examples of soluble supports include linear polymers such as poly(ethylene glycol), dendrimers, or fluorinated compounds which selectively partition into fluorine-rich solvents.

The support may take any physical form. The support may be a particle or bead, a film, a mesh, a tube, a cylinder, an optic fibre amongst others. The support may also be a fining on a particle or bead, a film, a mesh, a tube, a cylinder amongst others.

The support may be magnetic, or comprise a magnetic material. The support may be ferromagnetic or paramagnetic.

The support may be particle with or without an external coating. The particle may have a solid core of polymeric material or a core of metal or a mixture of both. The metal may be in metallic form or in salt form.

The support may be a polymer, such as a poly(styrene) or a polysaccharide, or the support may be a dendrimer, preferably a high generation dendrimer.

The support may be a metal, such as gold, or a metal oxide or other metal salt.

The support may be a glass, typically in the form of a fibre or a slide.

The support may be a semiconductor material, typically in the form of a wafer.

The support may be a chip, or other such surface, for use with an analytical device, for example an SPR (surface plasmon resonance) device.

Preferably the support is relatively inert. That is to say, the support should preferably have little or no affinity for the substance. The support can be coated with a material to minimise non-specific binding.

The term ‘support’ may also refer to a material having a rigid or semi-rigid surface which contains or can be derivatized to contain reactive functionalities which can serve for covalently linking a compound to the surface thereof. Such materials are well known in the art and include, by way of example, silicon dioxide supports containing reactive Si—OH groups, polyacrylamide supports, polystyrene supports, polyethyleneglycol supports, and the like. The support may be a support having a mixture of functionality. For example the support may have a polystyrene backbone grafted on to which is polyethyleneglycol. Such supports are available as Tentagel™. Such supports may take the form of small beads, pins/crowns, laminar surfaces, pellets, disks. Other conventional forms may also be used.

It will be appreciated that the support may have functional sites where a linker may be attached.

The precise ‘loading’ of the support, the number of available functional sites per unit mass, will depend on the exact nature of the support. The loading may be provided by the commercial supplier of that support. The loading can also be measured experimentally by any one of the methods that are known in the art, such as elemental analysis, ¹H and ¹³C NMR. The loading can also be determined from mass difference calculations derived from the addition or removal of a compound from the support. This may be accompanied by spectroscopic measurements, such as those based on the so-called ‘Fmoc count’.

For convenience, where the support is drawn herein, the support is shown attached to only one linker. However the actual number of functional groups on a support will be very much higher than this. A commercially available resin support such as aminomethylated polystyrene may have anywhere from 0.25-0.75 mmoleg⁻¹ amino functional groups. A support such as the Sepharose support CI-6B (an agarose-based support) may have a loading of around 24 μmoleg⁻¹.

### Linker

The compounds of the invention may be connected to the support through a linker. The linker may be a direct bond or a group such as an optionally substituted C₁₋₂₀ alkyl or optionally substituted C₅₋₂₀ aryl. The linker may be provided to assist analysis or to provide functionality that will allow cleavage of a compound from the support. The linker may also provide a structural or functional unit capable of interacting with a substance of interest.

The linker may be a cleavable linker that is capable of releasing the compound form the solid support. Alternatively the linker may a non-cleavable linker. The linker may be a flexible linker.

When a linker is cleaved to release a compound from the support, part of the linker structure may be included as a part of the released compound. Alternatively, the compound may be released without any part of the linker molecule included. The compound may be released leaving a functional group ‘stub’ such as a carboxylic acid group on the compound, or leaving a hydrogen on the compound. Linkers that are capable of the latter are referred to as traceless linkers.

Among the linkers that may be used in the compounds of the present invention are linkers based on Wang, HMPB, HMPA, Sieber amide, Rink amide, FMPB, DHP, chlorotrityl, hydrazinobenzoyl, sulfamylbutyrl, oxime, and MBHA amongst others. Such linkers are widely available from commercial sources. See, for example, the Novabiochem Catalog 2006/2007.

Alternatively, the linker may be a non-commercial linker.

It is also possible that the linking group is a simple functionality provided on the solid support, e.g. amine, and in this case the linking group may not be readily cleavable. This type of linking group is useful in the synthesis of collections which will be subjected to on-bead screening (see below), where cleavage is unnecessary. Such resins are commercially available from a large number of companies including NovaBiochem, Advanced ChemTech and Rapp Polymere. These resins include amino-Tentagel, and amino methylated polystyrene resin.

Linkers may be cleaved under a variety of conditions, and the linker chosen for use in the invention may

The linker may additionally include a spacer between the support and the linker functionality. The spacer may be included to avoid steric hindrance during the adsorption and desorption process. Typically, the spacer is a short, flexible alkyl group.

### Includes Other Forms

Included in the above are the well known ionic, salt, solvate, and protected forms of these substituents. For example, a reference to a substituent carboxylic acid (—COOH) in a compound of formula (I), (II), (III) or (IV) also includes the anionic (carboxylate) form (—COO⁻), a salt or solvate thereof, as well as conventional protected forms. Similarly, a reference to a substituent amino group in a compound of formula (I), (II), (III) or (IV) includes the protonated form (—N⁺HR¹R²), a salt or solvate of the amino group, for example, a hydrochloride salt, as well as conventional protected forms of an amino group. Similarly, a reference to a substituent hydroxyl group a compound of formula (I), (II), (III) or (IV) also includes the anionic form (—O⁻), a salt or solvate thereof, as well as conventional protected forms of a hydroxyl group.

### Isomers

Certain compounds may exist in one or more particular geometric, optical, enantiomeric, diasterioisomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

If the compound is in crystalline form, it may exist in a number of different polymorphic forms.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below, as well as its different polymorphic forms.

### Salts and Ions

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: acetic, propionic, succinic, gycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, toluenesulfonic, methanesulfonic, ethanesulfonic, ethane disulfonic, oxalic, isethionic, valeric, and gluconic. Examples of suitable polymeric anions include, but are not limited to, those derived from the following polymeric acids: tannic acid, carboxymethyl cellulose.

### Protected Forms

It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, “Protective Groups in Organic Synthesis” (T. Green and P. Wuts; 3rd Edition; John Wiley and Sons, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO.).

For example, a carboxylic acid group may be protected as an ester for example, as: a C₁₋₇ alkyl ester (e.g. a methyl ester; a t-butyl ester); a C₁₋₇ haloalkyl ester (e.g. a C₁₋₇ trihaloalkyl ester); a triC₁₋₇ alkylsilyl-C₁₋₇alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

Where reference is made to a group that is derived from an amino acid, where appropriate, the amino-, carboxy- or side chain-functionality may be protected. For the amino group the protecting groups may be selected from the group consisting of Fmoc, Boc, Ac, Bn and Z (or Cbz). The side-chain may also be protected as appropriate. The side chains protecting groups may be selected from the group consisting of Pmc, Pbf, OtBu, Trt, Acm, Mmt, tBu, Boc, ivDde, 2-ClTrt, tButhio, Npys, Mts, NO₂, Tos, OBzl, OcHx, Acm, pMeBzl, pMeOBz, OcHx, Born, Dnp, 2-Cl—Z, Bzl, For, and 2-Br—Z as appropriate for the side chain. The carboxy-group may be protected as an ester, such as a methyl ester.

### Preferences

Preferred compounds of the fourth aspect of the invention are described below. The preferences for the compounds of formula (III) and (IV) of the fourth aspect of the invention are also independently applicable to each compound formula (I) and (II) according to the collections of the first aspect of the invention.

References to R² are made only in relation to compound of formula (III) and (I).

The preferences are also independently applicable to components for use in the methods of the third, ninth and tenth aspects of the invention.

The preferences below may be combined in any combination as appropriate.

### Support

Preferably the support comprises a glass, gold, a polystyrene, a polysaccharide, a polyacrylamide or a poly(alkoxide). The support may be a polysaccharide, most preferably agarose.

### Linker

The linker may additionally include a spacer between the linker and the point of attachment. The spacer may be an optionally substituted C₁₋₂₀ alkyl, optionally substituted C₃₋₂₀ heterocyclyl or an optionally substituted C₅₋₂₀ aryl. The spacer may be a an optionally substituted C₁₋₆ alkyl group

The linker itself may be an analytical linker which may be removed from the support with the affinity fragment. Such linkers are well known in the art.

Preferably the linker, together with the support, is represented by the formula (V):

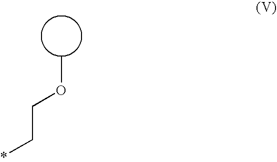

wherein the asterisk “*” is the point of attachment and the circle represents the support.

Where one of R^{1a} and R^{1b} is a group comprising a linker attached to a support, then the linker is preferably a linker derived from an aldehyde-functionalised linker. The linker, together with the support, may be derived from formyl polystyrene, tentagel acetal resin, 3-formylindolyl)acetamidomethyl polystyrene or Garner aldehyde functionalised amino-methylated polystyrene, amongst others.

Where one of R^{1a} and R^{1b} is a group comprising a linker attached to a support, preferably the linker is represented by formula (V).

Where R² is a group comprising a linker attached to a support, then the linker is preferably a linker derived from an amine-functionalised linker. The linker, together with the support, may be derived from amino-methylated polystyrene, 3-amino-phenoxymethyl polystyrene, aminomethyl NovaGel™, Tentagel™ amino ethyl, amino PEGA, [G 1,3]-aminodendrimer polystyrene, MBHA, amino-(4-methoxyphenyl)methyl polystyrene, Rink amide resin, hydroxylamine Wang resin, and sulfamyl resin amongst others.

Where R⁴ is a group comprising a linker attached to a support, then the linker is preferably a linker derived from a carboxy-functionalised linker. The linker, together with the support, may be derived from carboxypolystyrene and Tentagel™ carboxy resin amongst others.

### R^{1a}, R^{1b}, R², R³, R⁴, R⁵, R⁶ and R⁷

R^{1a}, R^{1b}, R², R³, R⁴, R⁵, R⁶ and R⁷ may be optionally substituted or optionally further substituted as appropriate.

The alkyl group may be a C₁₋₁₀ alkyl group, preferably a C₁₋₆ alkyl group.

The aryl group may be a C₅₋₂₀ aryl group, preferably a C₅₋₇ aryl group. Alternatively, the aryl group may be a C₁₀₋₂₀ aryl group.

The heterocyclyl group may be a C₅₋₂₀ heterocyclyl group, preferably a C₅₋₇ heterocyclyl group. Alternatively, the heterocyclyl group may be a C₁₀₋₂₀ heterocyclyl group.

Where two or more of the others of R^{1a}, R_{1b}, R², R³ and R⁴, together with the atoms to which they are bound, form a ring, the ring is preferably a C₅₋₂₀ heterocyclyl group. The C₅₋₂₀ heterocyclyl group may have a C₅₋₂₀ aryl substituent.

Preferably two of the others of R^{1a}, R^{1b}, R², R³ and R⁴, together with the atoms to which they are bound, form a ring. Where the two of the others are selected from R², R³, R⁴ and R^{1a} or R^{1b}, together the two may be referred to as a bidentate substituent.

Where the substituent R^{1a}, R^{1b}, R², R³ and R⁴ does not comprise a linker attached to a support, the substituent is optionally substituted C₁₋₂₀ alkyl, optionally substituted C₃₋₂₀ heterocyclyl or optionally substituted C₅₋₂₀ aryl. Preferably the alkyl or aryl group is substituted.

The optionally substituted C₁₋₂₀ alkyl, optionally substituted C₃₋₂₀ heterocyclyl or optionally substituted C₅₋₂₀ aryl group may contain an analytical label that allows the compound to be located and/or identified. The analytical label may be a group that provides a characteristic signal when analysed, e.g. by spectroscopic methods. In one embodiment the label is a fluorescent label. Additionally or alternatively, the label may be provided by one or more isotopes, including radioisotopes. This label may assist in detection and identification of products cleaved from the support by mass spectrometry, for example by providing unique isotope patterns. The label may also assist analysis by NMR, where an isotope in the label may increase the intensity of an observed signal in the NMR spectrum. Example isotopes for use in the label include, but are not limited to, ²H (D) and ¹³C. Such analysis allows the compound to be studied without the need for removal of a fragment form the support.

The label may include a functional group with a characteristic IR stretching frequency. The label may include a functional group that is capable of reacting with a reagent, the product of which reaction is capable of indicating that the corresponding compound is present. The reaction product may a coloured product allowing identification by eye.

The label may be fluorescent or luminescent, or coloured such that a support attached to the label will be visible to the eye. Such labels also allow the compound to be studied without the need for removal of a fragment form the support.

Where the compound comprises a cleavable linker, that linker may be cleaved to release a fragment for analysis. Cleavage strategies are described above in relation to linkers. Alternatively the label itself may be cleavable from the resin

Other labels will be known to those of skill in the art.

The aryl group may be fluorescent. The aryl group may be a pyrene. Preferably the pyrene is selected from the group:

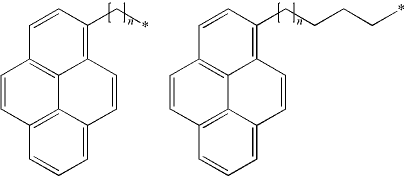

where n is 0 or 1 and the asterisk indicates the point of attachment.

The substituted C₁₋₂₀ alkyl, substituted C₃₋₂₀ heterocyclyl or substituted C₅₋₂₀ aryl group may be substituted with one or more substituents independently selected from the group consisting of: acetal, hemiacetal, alkoxy, ketal, hemiketal, oxo, thione, imino, formyl, halo, hydroxy, thiocarboxy, thiolocarboxy, imidic acid, hydroxyamic acid, thionocarboxy, ether, nitro, cyano, ether, nitro, nitroso, azido, cyanato, isocyanto, thiocyano, isothioctano, cyano, acyl, carboxy, ester, amido, amino, guanidino, tetrazoyl, imino, amidine, acylamido, ureido, acyloxy, thiol, disulfide, thioether, sulfoxide, sulfonyl, thioamido, sulfinyloxy, sulfate, sulfonamido, sulfonate, sulfamino, phosphino, phospho, phosphinyl, phosphonic acid, phosphonate, phosphate, phosphoric acid, phosphorous acid, phosphoramidite, phosphoramidate, silyl, oxysilyl, siloxy, oxysiloxy and sulfonamino. Additionally, an alkyl substituent may itself be substituted with an aryl or heterocyclyl group and vice versa.

Most preferably the substituted C₁₋₂₀ alkyl, substituted C₃₋₂₀ heterocyclyl or substituted C₅₋₂₀ aryl group is substituted with one or more substituents independently selected from the group consisting of: hydroxy, halo, nitro, sulfonic acid, sulfonamido, oxo, thione, carboxy, amino, boronic acid, amido, thioamido. Additionally, an alkyl substituent may itself be substituted with an aryl or heterocyclyl group and vice versa.

The preferred aryl and alkyl substituents may themselves be substituted with one or more substituents selected from the list of preferred substituents.

### R^{1a} and R^{1b}

Where R^{1a} and R^{1b} are not a group comprising a linker attached to a support, then R^{1a} and R^{1b} may both be hydrogen.

Preferably, R^{1a} is a substituent comprising a linker attached to a support

Where R^{1b} is not a substituent comprising a linker attached to a support, then preferably, R^{1b} is hydrogen.

Preferably, R^{1b} is hydrogen.

Where either of R^{1a} or R^{1b} is not a group comprising a linker attached to a support, then of R^{1a} or R^{1b} may be independently selected from the list of substituents given in the table below:

R^{1a} or R^{1b}

| | |
|---|---|
| 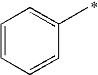 | 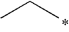 |
| —* | 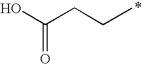 |

where the asterisk ‘*’ indicates the point of attachment.

### R²

Where R² is not a group comprising a linker attached to a support, then R² may be selected from the list of substituents given in the table below:

R²

| | |
|---|---|
| 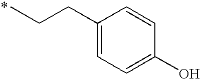 | 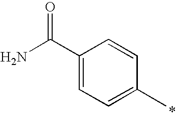 |
| 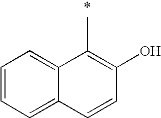 | 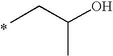 |
| 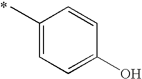 | 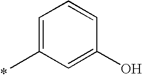 |
| 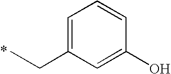 | 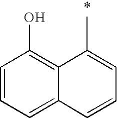 |
| 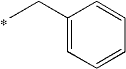 | 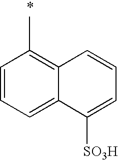 |
| 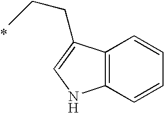 | 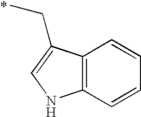 |
| 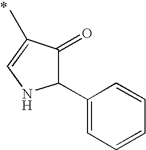 | 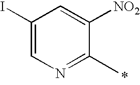 |
| 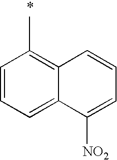 | 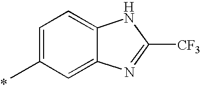 |
| 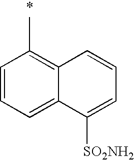 | 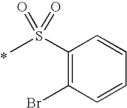 |
| 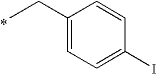 | 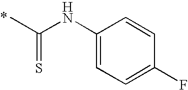 |
| 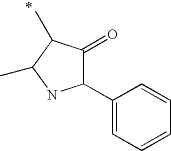 | 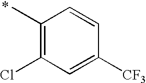 |
| 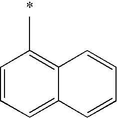 | 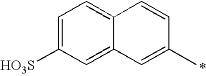 |
| 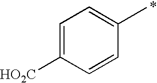 | 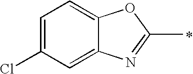 |
| 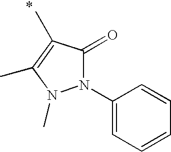 | 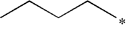 |
| 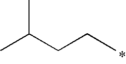 | 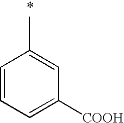 |
| 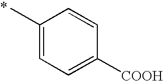 | 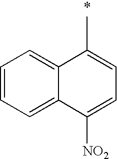 |
| 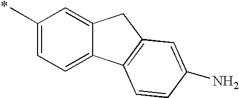 | 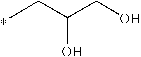 |
| 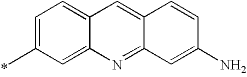 | 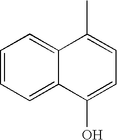 |
| 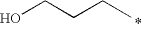 | 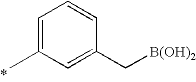 |
| 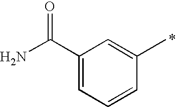 | 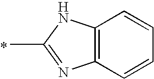 |
| 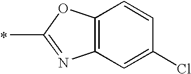 | 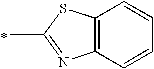 |
| 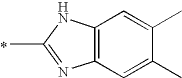 | 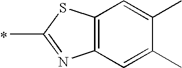 |
| 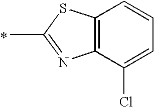 | 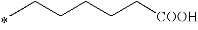 |
| 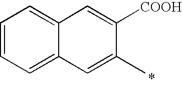 | 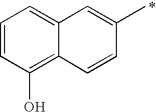 |
| 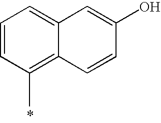 | 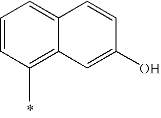 |
| 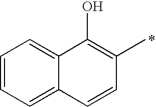 | 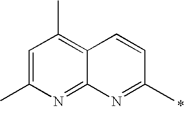 |
| 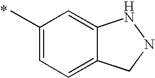 | 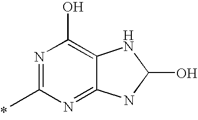 |
| 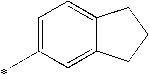 | 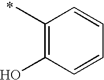 |
| 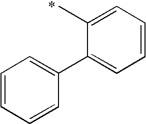 | 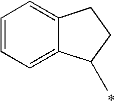 |
| 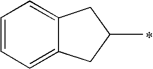 | 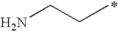 |
| 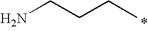 | 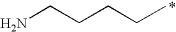 |
| 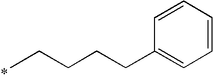 | 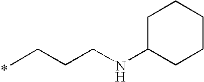 |
| 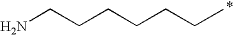 | 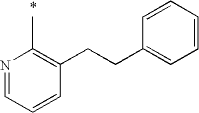 |
| 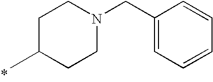 | 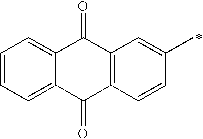 |
| 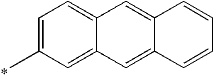 | 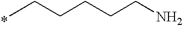 |
| 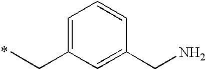 | 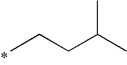 |
| 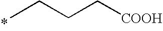 | 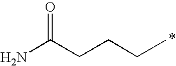 |
| 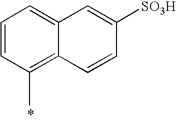 | 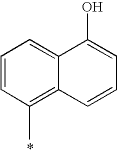 |
| 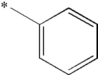 | 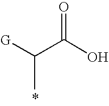 |

where the asterisk indicates the point of attachment.

In the table above G represents a side chain of an amino acid. For example, G is —H for glycine, and G is —CH₃ for alanine. G may be the side chain of any natural or non-natural amino acid. Preferably, the side chain is a side chain of alanine, arginine, asparagine, aspartic acid, cysteine, glutamic acid glutamine, glycine, histidine, isoleucine, leucine, lysine, methionine, phenylalanine, serine, threonine, tryptophan, tyrosine or valine. A R² amino acid may be derived from an L- or a D-amino acid.

Where R² is not a group comprising a linker attached to a support, then the most preferred substituents are selected from the list given in the table below:

R²

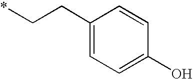

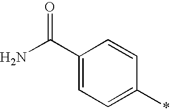

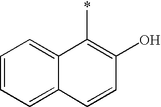

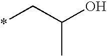

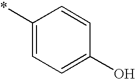

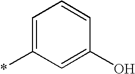

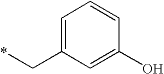

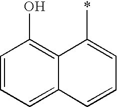

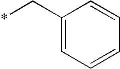

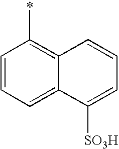

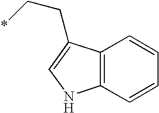

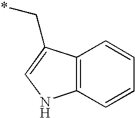

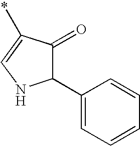

where the asterisk indicates the point of attachment.

### R³

Where R³ is not a group comprising a linker attached to a support, then R³ may be selected from the list given in the table below:

R³

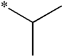

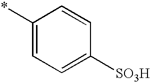

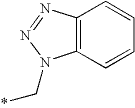

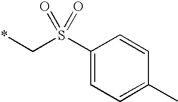

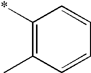

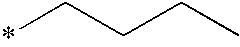

where the asterisk ‘*’ indicates the point of attachment.

### R⁴

Where R⁴ is not a group comprising a linker attached to a support, then R⁴ may be selected from the list of substituents given in the table below:

R⁴

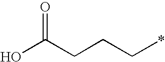

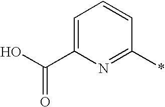

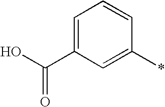

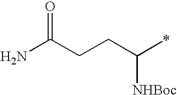

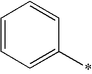

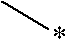

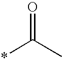

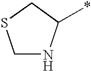

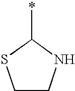

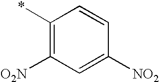

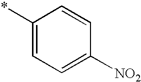

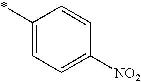

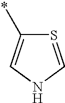

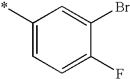

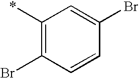

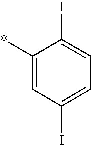

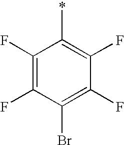

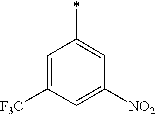

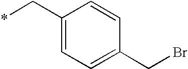

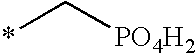

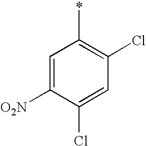

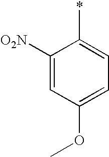

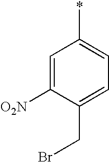

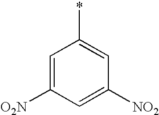

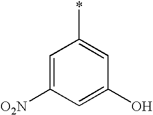

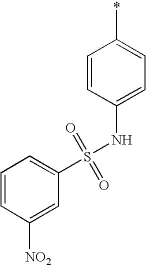

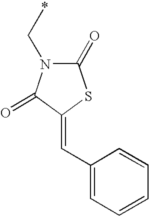

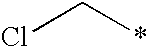

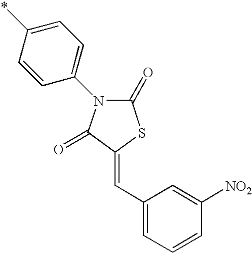

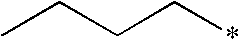

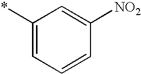

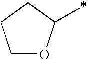

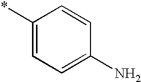

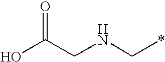

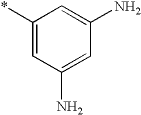

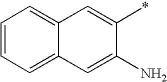

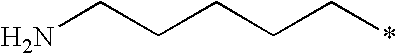

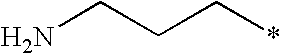

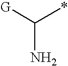

where the asterisk ‘*’ indicates the point of attachment.

In the table above G represents a side chain of an amino acid. For example, G is —H for glycine and G is —CH₃ for alanine. G may be the side chain of any natural or non-natural amino acid. Preferably, the side chain is a side chain of alanine, arginine, asparagine, aspartic acid, cysteine, glutamic acid glutamine, glycine, histidine, isoleucine, leucine, lysine, methionine, phenylalanine, proline, serine, threonine, tryptophan, tyrosine or valine.

Where R⁴ is not a group comprising a linker attached to a support, then the most preferred substituents are selected from the list in the table below:

R⁴

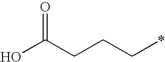

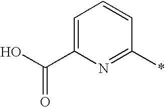

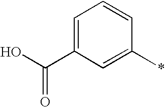

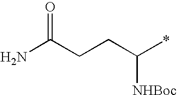

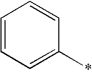

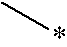

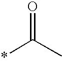

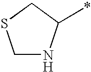

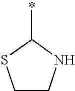

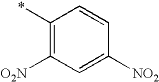

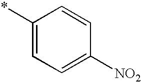

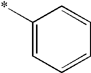

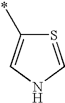

where the asterisk ‘*’ indicates the point of attachment.

### Collections

The present invention relates to libraries, or collections, of compounds. Each member of the collection is represented by a single one of the formulae (I) or (II). The diversity of the compounds in a library may reflect the presence of compounds differing in the identities of one or more of the substituent groups. The number of members in the library depends on the number of variants, and the number of possibilities for each variant. For example, if it is the substituents R², R³ and R⁴ are varied, with 3 possibilities for each substituent, the library will have 27 compounds (3×3×3). A library may comprise more than 1,000, 5,000, 10,000, 100,000 or a million compounds, which may be arranged as described below. Alternatively, the library may contain 96 compounds, or a multiple thereof.

Collections of compounds of formulae (I) and (II) may be held in discrete volumes of solvents, e.g. in tubes or wells. Alternatively the collection may be held as discrete particles, where appropriate, or as discrete gels. Collections of compounds are preferably bound at discrete locations, e.g. on respective pins/crowns or beads. The collection of compounds may be provided on a plate which is of a suitable size for the library, or may be on a number of plates of a standard size, e.g. 96 well plates. If the number of members of the library is large, it is preferable that each well on a plate contains a number of related compounds from the library, e.g. from 10 to 100. One possibility for this type of grouping of compounds is where only a subset of the substituents are known and the remainder are randomised; this arrangement is useful in iterative screening processes (see below). The library may be presented in other forms that are well-known.

### Preparation of Compounds

The compounds of the invention are typically prepared using multi component reactions. The most preferred reaction types for use in the present invention are Ugi- and Passerini-based reactions.

Generally, the Ugi reaction comprises the step of contacting an aldehyde-functionalised reagent, a carboxylic acid-functionalised reagent, an amine-functionalised reagent and an isonitrile-functionalised reagent, typically in one reaction vessel. Generally, the Passerini reaction comprises the step of contacting an aldehyde-functionalised reagent, a carboxylic acid-functionalised reagent and an isonitrile-functionalised reagent, typically in one reaction vessel.

Multicomponent reactions such as the Ugi reaction possess a number of distinct advantages over more conventional ‘2-component’ methods. Firstly, multi-component reactions allow for a greater diversity of ligands by incorporating three or four (or more) reactants, each of which can be varied systematically to produce a huge variety of subtle changes to the final ligand structure. The apparent ease of the rapid chemical substitution process lends itself to combinatorial techniques thereby hugely increasing the “chemical space” that can be readily investigated in a relatively short period of time—in other words it is possible to generate a very large number of compounds in a few simple steps. Hence it is possible to explore chemical hypotheses by casting a ‘wider net’ and provides a viable alternative to the more traditional ‘shot-gun’ approach based on a limited set of highly diverse compounds. A short survey of the number of commercially available compounds suitable for this particular multicomponent chemistry reveals the potential for this approach to increase scaffold diversity and application as novel affinity adsorbents (Table 1). Secondly, the “one-pot” nature of multi-component reactions offers considerable saving on time, reagent costs and purification techniques, thus making it possible to probe a larger number of chemical hypotheses more efficiently. The promptness of reagent delivery and requirement for chemical diversity are addressed within a single synthesis step. The Ugi reaction is a good example of convergent synthesis, allowing multiple bond formation to occur between the various components without the need to isolate and identify any chemical intermediates and thus making this procedure highly desirable for combinatorial library synthesis.

TABLE 1
Current list of commercially available Ugi reaction components
from the Available Chemicals Directory (ACD)

| Functional group | | Commercial availability |
|---|---|---|
| Primary/secondary amines | R—NH₂ | 95,398 |
| Aldehydes | R—CHO | 10,982 |
| Isonitrile | R—NC | 644 |
| Carboxylic acid | R—COOH | 2,158 |

The difficult issue of variable reactivity of the chemical constituents exerts a far less significant impact on the final compound yield for the Ugi reaction: Certain amines such as tryptamine and tyramine exhibit hyper-reactivity when coupled to triazine-activated agarose (unpublished work, Hussain 2001), tending to result in undesirable bi-substituted reaction products. However, in the multi-component reaction or Ugi reaction, the mechanism of the reaction is such that the question of amine reactivity is less important as the reaction requires equimolar quantities of each of the four components to go to completion. If a reactant is particularly unreactive, the reaction will not proceed to any significant degree. Therefore, there are no ‘partial products’ or undesired by-products formed.

An additional advantage of using the Ugi chemistry for ligand design is the potential for the scaffold to mimic a native dipeptide bond. The difference in the calculated interatomic distances between the O1-N—O2 in the native dipeptide bond as compared to the Ugi scaffold are less than 1.0 Å between all three atoms suggesting that this scaffold may have the ability to correctly mimic a native dipeptide bond. Also, note the presentation of the R4 (carboxylic acid) and R2 (amine) moieties which both protrude away from the scaffold and hence the surface of the chromatographic matrix. These two functional groups therefore present an exploitable binding site for target interaction.

### Ugi Reaction

According to one aspect of the invention there is provided a method for the preparation of a compound according to formula (III). The compound may be prepared using the multicomponent Ugi reaction. According to the present invention the process comprises the step of contacting components A, B, C and D together, wherein
A is R^{1a}COR^{1b};
B is R²—NH₂;
C is R³—NC;
D is R⁴—COOH; and
one of R^{1a}, R^{1b}, R², R³ and R⁴ is a group comprising a linker attached to a support,
and the others R^{1a}, R^{1b}, R², R³ and R⁴ are independently selected from optionally substituted C₁₋₂₀ alkyl, optionally substituted C₃₋₂₀ heterocyclyl or optionally substituted C₅₋₂₀ aryl, and R^{1a}, R^{1b} and R² are additionally selected from hydrogen, and R² is additionally further selected from —S(═O)R⁵ and —C(═S)NR⁶R⁷, wherein R⁵, R⁶ and R⁷ are independently optionally substituted C₁₋₂₀ alkyl, optionally substituted C₃₋₂₀ heterocyclyl or optionally substituted C₅₋₂₀ aryl,
or, optionally, two or more of the others of R^{1a}, R^{1b}, R², R³ and R⁴ are connected.

In one embodiment the compounds of the reaction may be prepared by combining all of the reagents in one reaction vessel. Alternatively, the amine and aldehyde/ketone component (B and A respectively) may be pre-reacted, thereby to form an imine intermediate, prior to the addition of the other, carboxylic acid and isonitrile reagents (D and C respectively). Preferably, these reactions are performed in one pot.

Where two or more of R^{1a}, R^{1b}, R², R³ and R⁴ are connected, the corresponding reagent may be referred to as a bidentate reagent, as when two substituents are connected, or a tridentate reagent, as when three substituents are connected.

Where A, B, C or D contains an additional functional group, this group may be in a protected form. Example protecting groups are described above. This protecting group may be removed once the scaffold product has been formed. For example, a reagent B may have a carboxylic acid group. This group may be protected as a free acid (COO⁻) or as an ester (COOMe), which may be hydrolysed to the acid when required. A reagent D may have an amino group (—NH₂). This group may be protected with Fmoc (—NHFmoc). This protecting group may be removed later with e.g. pyridine or DBU.

Amino acid components may be used as reagents B and D. Suitably protected forms of amino acids, where the amino-, carboxy- or side chain-functionality is protected as appropriate, are well known in the art and are readily available form commercial sources e.g. Aldrich and Novabiochem.

Where A is a group comprising a linker attached to a support, then one of R^{1a} or R^{1b} may be a formyl polystyrene, tentagel acetal resin, 3-formylindolyl)acetamidomethyl polystyrene or Garner aldehyde functionalised amino-methylated polystyrene, amongst others.

The preferences for R^{1a}, R^{1b}, R², R³, R⁴, R⁵, R⁶ and R⁷ are the same as those given for the compounds of formula (I) and (III) above.

The preferences for the ligand and support are the same as those given in relation to the linkers and supports for the compounds and the collections described above.

According to the third aspect of the invention, the is provided a method for preparing a compound identified as having affinity for a substance. In one embodiment, the step comprises contacting components A, B, C and D together. One of these components may be a structural or functional analogue of the linker of the library member. For instance, where the linker comprises an aryl group, the analogue may include an aryl group.

### Passerini Reaction

According to one aspect of the invention there is provided a method for the preparation of a compound according to formula (IV). The compound may be prepared using the multicomponent Passerini reaction. According to the present invention the process comprises the step of contacting components A, C and D together, wherein
A is R^{1a}COR^{1b};
C is R³—NC;
D is R⁴—COOH; and
one of R^{1a}, R^{1b}, R³ and R⁴ is a group comprising a linker attached to a support,
and the others of R^{1a}, R^{1b}, R³ and R⁴ are independently selected optionally substituted C₁₋₂₀ alkyl, optionally substituted C₃₋₂₀ heterocyclyl or optionally substituted C₅₋₂₀ aryl, and R^{1a} and R^{1b} are additionally selected from hydrogen,
or, optionally, two or more of the others of R^{1a}, R^{1b}, R³ and R⁴ are connected.

Where two or more of R^{1a}, R^{1b}, R³ and R⁴ are connected, the corresponding reagent may be referred to as a bidentate reagent, as when two substituents are connected, or a tridentate reagent, as when three substituents are connected.

Where A, C or D contains an additional functional group, this group may be in a protected form. Example protecting groups are described above. This protecting group may be removed once the scaffold product has been formed. For example, reagent D may have an amino group (—NH₂). This group may be protected with Fmoc (—NHFmoc). This protecting group may be removed later with e.g. pyridine or DBU.

The preferences for R^{1a}, R^{1b}, R³ and R⁴ are the same as those given for the compounds of formula (II) and (IV) and above.

The preferences for the ligand and support are the same as those given in relation to the linkers and supports for the compounds and the collections described above.

### Preparation of Collections

The methods described above for the preparation of compounds of formula (III) and (IV) are applicable to the preparation of collection of compounds of formula (I) and (II): The members of the collection may be prepared in parallel using, for instance using techniques common in the art of combinatorial chemistry. These steps may be automated using techniques well known in the art.

### Analysis

Compounds of formula (III) and (IV) may be analysed by IR, NMR (gel-phase and magic angle spinning (MAS) techniques) and elemental analysis, amongst others. Where the linker is a cleavable linker, the linker may be cleaved to release a compound from the support. The released compound may be analysed using techniques common in the art e.g. LC-MS, HPLC, NMR, elemental analysis, IR, TLC and gravimetric analysis to establish the identity and amount of the compound, and consequently the identity and amount of material on the solid support.

Individual members of a collection may also be analysed by the techniques described above. The analysis of the members may automated.

As discussed above in relation to linkers and the groups R^{1a}, R^{1b}, R², R³ and R⁴, any one of these may contain an analytical marker to assist identification and quantification of a reaction method and the identify and quantity of a reaction product.

### Use of Compounds and Collections

The compounds and collections described herein may be used in methods of purification. The compounds may also be incorporate into analytical or diagnostic devices.

The compounds may be used to identify ligands for a conformational form of a substance. For example, the compounds may be used to identify ligands for the G-quadruplex structure on a section of telomere-like DNA. Preferably such compounds would be selective for one conformational form over another conformational form of that substance.

The binding between a substance and a ligand may be detected in any one of numerous ways. The substance itself may have a label that allows it to be identified.

The compounds in a collection may be spatially arranged e.g. on a surface or between the wells of a well plate.

The present invention also relates to a method of screening the compounds of formula III and IV to discover biologically active compounds. The screening can be to assess the binding interaction with nucleic acids, e.g. DNA or RNA, or proteins, or to assess the affect of the compounds against protein-protein or nucleic acid-protein interactions, e.g. transcription factor DP-1 with E2F-1, or estrogen response element (ERE) with human estrogen receptor (a 66 kd protein which functions as hormone-activated transcription factor, the sequence of which is published in the art and is generally available). The screening can be carried out by bringing the target macromolecules into contact with individual compounds or the arrays or libraries described above, and selecting those compounds, or wells with mixtures of compounds, which show the strongest effect.

This effect may simply be the cytotoxicity of the compounds in question against cells or the binding of the compounds to nucleic acids. In the case of protein-protein or nucleic acid-protein interaction, the effect may be the disruption of the interaction studied.

Another aspect of the present invention relates to the use of compounds of formula III and IV in diagnostic methods. A compound of formula III and IV which binds to an identified sequence of DNA or a protein known to be an indicator of a medical condition can be used in a method of diagnosis. The method may involve passing a sample, e.g. of appropriately treated blood or tissue extract, over an immobilised compound of formula III and IV, for example in a column, and subsequently determining whether any binding of target DNA to the compound of formula III and IV has taken place. Such a determination could be carried out by passing a known amount of labelled target DNA known to bind to compound III and IV through the column, and calculating the amount of compound III and IV that has remained unbound.

A further aspect of the present invention relates to the use of compounds of formula III or IV in target validation. Target validation is the disruption of an identified DNA sequence to ascertain the function of the sequence, and a compound of formula III or IV can be used to selectively bind an identified sequence, and thus disrupt its function, i.e. functional genomics. Collections of compounds of formula (I) and (II) may be used in a similar manner.

The present invention also provides for the purification of contaminants from a mixture. A compound may be capable of immobilising a contaminant in a mixture. Removal of the contaminant from the mixture thereby purifies the mixture. Such a method may involve the use of several compounds, each having a affinity for a different contaminant.

The method may involve contacting a mixture with the several compounds in one step, thereby removing multiple contaminants at the same time. This may improve mixture purification times, and hence increase throughput.

A library of compounds may be obtained from a commercial source, or may be prepared according to the methods described herein.

### DEFINITIONS

### Substances

The present invention provides for the purification of a substance from a mixture as well as methods for the identification of affinity ligands for a substance.

The substance may be any entity which it is desirable to isolate from a mixture. The substance may also be any entity which it is desirable to identify a compound capable of binding thereto.

The substance may be a small or large organic molecule (<500 Daltons and ≧500 Daltons respectively), a macromolecule, a polymer such as a nucleic acid or peptide, or a complex entity such as a cell, such as a bacterium, or a virus.

The substance may be a compound having biological activity. The substance may have structural, regulatory, or biochemical functions of a naturally occurring molecule. The substance may be a metabolite, a drug, an enzyme, a messenger or the like.

Preferably the substance is a nucleic acid, peptide, saccharide, or polyketide or lipid, including glycosilated versions.

Preferably the substance may be an enzyme inhibitor, regulatory enzyme, hormone-binding proteins, vitamin-binding proteins, receptors, lectins and glycoproteins, RNA and DNA, bacteria, viruses and phages, mycoplasmas, cells and genetically engineered protein products (e.g. HIS-tag conjugated proteins) derived from natural and artificial sources.

### Nucleic Acid and Peptide

Peptides includes polypeptides such as oligopeptides, ribosomal peptides, nonribosomal peptides, peptones and post-translationally modified forms thereof, as well as fragments variants and derivatives of these.

A peptide may be an enzyme, antibody or receptor, amongst others. The peptide may be any size. The peptide may be a polypeptide. Polypeptides typically comprise ten or more amino acid residues.

The term “antibody” is used in the broadest sense and specifically covers single monoclonal antibodies (including agonist and antagonist antibodies) and antibody compositions with polyepitopic specificity. The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly et al., supra; Morrison et al., Proc. Natl. Acad. Sci. U.S.A. 81:6851 (1984)).

The peptide may be a mammalian polypeptide, preferably a human polypeptide, or a polypeptide having high sequence identity with a human polypeptide (e.g. >70%, >80%, >90%, >95% identity).

Examples of mammalian polypeptides include molecules such as, e.g., rennin; a growth hormone, including human growth hormone or bovine growth hormone; growth-hormone releasing factor; parathyroid hormone; thyroid-stimulating hormone; lipoproteins; 1-antitrypsin; insulin A-chain; insulin B-chain; proinsulin; thrombopoietin; follicle-stimulating hormone; calcitonin; luteinizing hormone; glucagon; clotting factors such as factor VIIIC, factor IX, tissue factor, and von Willebrands factor; anti-clotting factors such as Protein C; atrial naturietic factor; lung surfactant; a plasminogen activator, such as urokinase or human urine or tissue-type plasminogen activator (t-PA); bombesin; thrombin; hemopoietic growth factor; tumor necrosis factor-alpha and -beta; antibodies to ErbB2 domain(s) such as 2C4 (WO 01/00245; hybridoma ATCC HB-12697), which binds to a region in the extracellular domain of ErbB2 (e.g., any one or more residues in the region from about residue 22 to about residue 584 of ErbB2, inclusive); enkephalinase; mullerian-inhibiting substance; relaxin A-chain; relaxin B-chain; prorelaxin; mouse gonadotropin-associated peptide; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as brain-derived neurotrophic factor (BDNF), neurotrophin-3, -4, -5, or -6 (NT-3, NT-4, NT-5, or NT-6), or a nerve growth factor such as NGF; cardiotrophins (cardiac hypertrophy factor) such as cardiotrophin-1 (CT-1); platelet-derived growth factor (PDGF); fibroblast growth factor such as aFGF and bFGF; epidermal growth factor (EGF); transforming growth factor (TGF) such as TGF-alpha and TGF-beta, including TGF-1, TGF-2, TGF-3, TGF-4, or TGF-5; insulin-like growth factor-I and -II (IGF-I and IGF-II); des(1-3)-IGF-I (brain IGF-I); insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha, -beta, and -gamma; a serum albumin, such as human serum albumin (HSA) or bovine serum albumin (BSA); colony stimulating factors (CSFs), e.g., M-CSF, GM-CSF, and G-CSF; interleukins (ILs), e.g., IL-1 to IL-10; anti-HER-2 antibody; Apo2 ligand (Apo2L); superoxide dismutase; T-cell receptors; surface-membrane proteins; decay-accelerating factor; viral antigens such as, for example, a portion of the AIDS envelope; transport proteins; homing receptors; addressins; regulatory proteins; antibodies; and fragments of any of the above-listed polypeptides.

Preferred substances for use in the present invention are blood proteins, particularly clotting proteins and most particularly Factor VII and Factor VIII, as well as fragments, variants and derivatives thereof.

In alternative embodiments, the substance may be an immunoglobulin, preferably IgG as well as fragments, variants and derivatives thereof.

Nucleic acids include DNA, RNA as well as the artificial forms PNA, LNA, GNA and TNA. The polynucleotide may include modified bases and/or a modified backbone. The nucleic acid may be any size.

The nucleic acid may be a sense or an antisense sequence.

The DNA may be mtDNA, cDNA, plasmid, cosmid, BAC, YAC, or HAC.

The RNA may be mRNA, piRNA, tRNA, rRNA, ncRNA, sgRNA, shRNA, siRNA, snRNA, miRNA, snoRNA, or LNA.

### Mixture

The term “mixture” may refer to any biological sample that may contain the substance of interest. A mixture can be a sample of biological fluid, such as whole blood or whole blood components including red blood cells, white blood cells, platelets, serum and plasma, ascites, urine, vitreous fluid, lymph fluid, synovial fluid, follicular fluid, seminal fluid, amniotic fluid, milk, saliva, sputum, tears, perspiration, mucus, cerebrospinal fluid, and other constituents of the body that may contain the analyte of interest, as well as tissue culture medium and tissue extracts such as homogenized tissue, and cellular extracts. Preferably, the sample is a body sample from any animal, but preferably is from a mammal, more preferably from a human subject. Most preferably, such biological sample is from clinical patients. The preferred biological sample herein is serum, plasma or urine, more preferably serum, and most preferably serum from a clinical patient.

A mixture may contain a contaminant. The contaminant is a material that is different from the desired substance. The contaminant may be a variant of a desired polypeptide (e.g. a variant of the desired polypeptide) or another polypeptide, nucleic acid, etc.

### Elution

A substance that is bound or otherwise associated with a compound (which may be referred to as an affinity ligand) may be removed from the compound using an elutant. The elution mixture is intended to disrupt the interaction between the support-bound ligand and the substance. The elution mixture may be chosen to disrupt hydrogen bonding interactions, electrostatic interactions and hydrophobic interactions between ligand and substance.

An “elution buffer” may be used to elute the substance of interest from the compound. The conductivity and/or pH of the elution buffer is/are such that the substance of interest is eluted from the support.

An elutant may be used as part of a method for studying the dissociation parameters of the substance and the compound. In such cases, the release of the substance over time form the compound is monitored.

Techniques for the separation of a substance from an affinity ligand are well known in the art.

### Analysis

There are many ways for determining whether an immobilised ligand is associated with a substance.

Where there compound is spatially separated from other compounds, the mixture which originally contained the substance may be removed, and the compound subsequently washed with an elution mixture to thereby remove the substance. That elution mixture may then be analysed to determine whether the substance is present and the degree to which it is present.

However, for the collections of the invention, such analysis may be impractical or impossible given the spatial arrangement of individual members of the collection.

In one embodiment, the substance may be radiolabelled. After the collection is washed to remove excess mixture, the collection may be analysed to determine the location and intensity of the radiation, thereby indicating the ligand to which the substance has bound and the degree to which it has bound.

In another embodiment, either the substance or the ligand may be labelled. The signal generated by the label may be quenched due to the association of the ligand with the substance. The addition of a test substance that competes with and displaces a substance from a preformed association complex will result in the generation of a signal above background. In this way, test substances that disrupt substance/ligand interaction can be identified.

Alternatively, a substance bound to a ligand may be detected using an ELISA-type assay.

The interaction of a compound with a substance, specifically a peptide, may also be determined using the Bradford protein assay.

These and other techniques are well known in the art.

### Separation

The present invention provides a method for separating a substance from a mixture according to the aspect of the invention. The mixture is contacted with a compound of the invention thereby to immobilise the substance in the mixture to the compound. The substance-depleted mixture may then be removed.

The substance may a contaminant. Alternatively, the substance may be a molecule of interest. The molecule of interest may be collected from the compound by treating the compound with an elutant.

Where the substance is a contaminant, the method results in the purification of the mixture. By purifying a mixture of one or more contaminants, it is meant increasing the degree of purity of a compound of interest in the composition by removing (completely or partially) at least one substance from the composition. A “purification step” may be part of an overall purification process resulting in a “homogeneous” composition, which is used herein to refer to a composition comprising at least about 70% by weight of the compound of interest, based on total weight of the composition, preferably at least about 80% by weight.

### Separation Apparatus

The compounds described herein may be incorporated into an apparatus for use in the purification of mixtures. The apparatus may be used to purify the mixture by immobilising a contaminant or alternatively by immobilising a desired substance, which may then be released from the apparatus at a later point.

The separation apparatus may take the form a chromatographic column which is packed with the appropriate compound. Alternatively, the apparatus may comprise a filter bed, where the bed includes the appropriate compound.

Within an apparatus, the compounds may be discrete particles or they may be bound to a surface or held in a porous matrix.

Other types of apparatus including an affinity ligand will be apparent to those of skill in the art.

### Experimental

### Materials

All chemicals were of reagent grade unless otherwise stated. Tyramine, 4-aminobenzamide, glutaric acid, 2,4-pyridine dicarboxylic acid, isophthalic acid, Boc-Glutamine, acetic acid, benzylamine, acetaldehyde, isopropyl isocyanide, isocyano-cyclohexane, epichlorohydrin, sodium periodate, sodium phosphate dibasic, ethylene glycol, sodium chloride, 1-pyrene methylamine and 1-pyrene butyric acid were all obtained from Sigma-Aldrich (Gillingham, UK). 1-amino-2-naphthol, 4-aminophenol, 3-aminophenol, amino-8-naphthol, benzoic acid and sodium hydroxide were obtained from Acros Organics, (Loughborough, UK). 4-hydroxybenzylamine was obtained from Chontech, Inc (Waterford, USA). Boc-Glycine and 1-amino-2-propanol was obtained from Fluke (UK). Ethanol, methanol, dichloromethane and propan-2-ol were all obtained from Fisher Chemicals, UK. Cross-linked agarose (Sepharose CL-6B) was purchased from G. E. Healthcare (Uppsala, Sweden). Human IgG (≧95% pure derived from pooled human serum) was obtained from Sigma (Dorset, UK) whilst hFab and Fc (≧95% pure derived from human plasma) was purchased from Calbiochem (Nottingham, UK). Polypropylene columns (0.8×6.0 cm) and frits were purchased from Varian (Oxford, UK). The 96-well standard microtitre plates and Coomassie Plus™ protein assay reagent (Bradford assay) for protein concentration determination were purchased from Corning Incorporated (Fisher Scientific UK) and Pierce (UK) respectively.

### Instrumentation

Ligand synthesis was performed using a Hybaid Maxi 14 hybridisation oven (Thermo Electron, UK). Total Protein concentration was determined using the Coomassie Plus™ protein assay reagent by measuring the absorbance of samples at wavelength (595 nm) using a Opsys MR plate reader from Dynex Technologies. Molecular images were obtained using the Molegro Virtual Docker 2007 software MVD v2.0.0 from Molegro ApS—Bioinformatic Solutions (Denmark). 1H and 13C nuclear magnetic resonance (NMR) spectra were performed using a Joel JNM Lambda LA400 FT NMR spectrometer. Mass spectra were recorded on AEI MS30 or AEI MS50 mass spectrometers in electron impact mode in the Chemical Laboratory, University of Cambridge, UK. Fluorescence studies were performed using an Olympus CX40 microscope, a Nikon EFD-3 filter (λex=330-380 nm), a Nikon mercury 100W lamp and a Kodak DC290 zoom digital camera.

### Methods

### Identification of Affinity Ligands for IgG

A collection of compounds was prepared to identify possible affinity ligands for IgG. The collection of compounds was based around a scaffold prepared by reacting an aldehyde-functionalised linker an aldehyde functionalized linker attached to a support with a carboxylic acid, an amine and an isonitrile in an Ugi multicomponent reaction. The products were then screened for their ability to bind IgG.

### Linker and Support Preparation

The matrix support Sepharose CL-6B (resin 2, scheme 1) is supplied as highly cross-linked, porous beads, 95 μM mean particle size, possessing primary terminal hydroxyl groups throughout the polymer network. The beads can be further modified by the addition of a ligand spacer arm as shown in the scheme below:

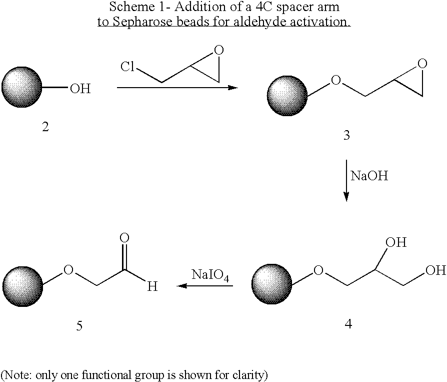

Sepharose beads were initially treated with epichlorohydrin in the presence of NaOH to yield epoxy-activated resin 3. The degree of activation achieved can be precisely controlled by the quantity of NaOH added at this step. The ‘epoxide activation assay’ requires the incubation of 3 with Na₂S₂O₃ and then titration against 0.1 M HCl revealing the epoxide content of the beads to within 1 μmol g⁻¹ of resin. When 3 was further treated with freshly prepared 5M NaOH, the epoxide form opens to generate the diol form 4. The latter was then subjected to 0.1M Nal₄ resulting in the cleavage of the diol form to leave the final aldehyde-activated resin 5.

### Epoxide Activation and Assay Determination

A sample of Sepharose beads (200 g) (resin 2, scheme 1) was poured into a grade 2 sinter-glass funnel and allowed to drain until a ‘settled gel’ consistency was obtained. This sample was weighed into a beaker and slurried to 50% bead/water v/v using sterile deionised water (200 ml). The slurry was then poured back into the sinter-glass funnel and washed thoroughly with water (5×400 ml) ensuring that the resin was well stirred before applying a vacuum and thus enabling filtration to occur. The last wash was left to drain thoroughly under gravity (10 mins) without applying a vacuum until a ‘settled’ gel' consistency was obtained again. The washed resin was slurried in water (100 mL) and transferred to a 500 mL duran bottle. 10 M NaOH (8 mL) was added to the slurry and left to stir at R.T. for 1 h. The temperature was then raised to 34° C. and fresh epichlorohydrin (14 mL) was added to the reaction mixture. The reaction mixture was maintained at 34° C. with gentle stirring for a period of 3 h. After this period, the contents of the duran bottle were poured into a grade 2 sinter-glass funnel and washed with deionised water (5×400 ml) to give the epoxide-activated resin (Residual epichlorohydrin was treated with NaOH for 24 h before safe waste disposal). Once settled, the resin was tested for its epoxide density by applying the epoxide activation assay previously mentioned above. A typical activation level of 24.0 μmol/g (settled gel) was obtained as measured by titration with 1.3M Na2S2O3.

### Cis-Diol Activation

The epoxide-activated resin (resin 3, scheme 1) (60 g) was treated with 5M NaOH (60 mL) and left to gently stir overnight at 34° C. This base-catalysed procedure gradually hydrolyses the epoxide ring resulting in the formation of a cis-diol reaction product 4.

### Aldehyde Activation

The diol-activated resin 4 (56 g) was then treated with 0.1M NaIO4 (100 ml) and left to stir at 30° C. for 3 h. This procedure causes the cleavage of the cis-diol, leaving a terminally functionalised aldehyde group. It is known that reactive aldehydes exposed to the air are prone to oxidation therefore the resin was immediately prepared for ligand library generation.

### Preparation of the Compound Collection

In order to generate a large number of ligands simultaneously, we employed the use of a Captiva™ 96-well block (supplied by Varian, UK) which contains a 20 μm polypropylene frit at the bottom of each well. This chemically-resistant block system thereby constituted the reaction vessel and the subsequent storage facility at the end of the final reaction.

A sample of the aldehyde-activated resin (resin 5, scheme 1) (36 g) was subjected to a series of washes of increasing methanol concentration, starting with 10% methanol and finishing with 100% methanol at 10% increments. This step is required as agarose beads may be subject to degradation if immediately placed in 100% methanol without gradually displacing the water absorbed by the resin. The methanol-saturated resin (36 g) was then slurried in 100% methanol (36 ml) and placed on a shaker with gentle shaking to prevent the resin from settling. A 1 ml Gilson pipette tip was cut off at approximately 2 mm from the end to allow for the easy transfer of 1 ml slurry aliquots into the 48 wells of the reaction block (8×6). The flexible end-cap mat was removed at this stage to allow the solvent to completely drain through and thus allow the resin to settle in the block. The end-cap mat was then firmly replaced in position at the bottom of the block.

A fixed concentration of the first pre-selected amine component (5× molar excess, in methanol) and volume (0.25 ml) was added down the first column of six wells (1, from A-F). A second different amine component was added down the second column (2, A-F) as mentioned above. This procedure was repeated until a total of eight different amines had been added to each column (see below for library component structures). The top cap-mat was then firmly attached to the block and allowed to shake for 1 h at 200 rpm. This procedure allowed the amine component to become completely mixed with the supplied resin sample.

Similarly, a fixed concentration of the first pre-selected carboxylic acid component (5× molar excess, in methanol) and volume (0.25 ml) was added across the first row (A, from 1-8). A second different carboxylic acid component was added down the second row (B, 1-8). This procedure was repeated until a total of six different carboxylic acids have been added across each of the six rows (see below for library component structures). Finally, a fixed aliquot (0.25 ml) of the isopropyl isocyanide component (5× molar excess, in methanol) was pipetted into each of the 48 wells. Therefore, for the construction of a 2D library array, only two of the four possible components involved in the Ugi reaction were varied.

The upper cap-mat was then firmly fixed to the top of the reaction block. The entire block was then placed in an incubation oven with a shaking platform (200 rpm) for 48 h at 50° C. At the end of the reaction period, the lower and upper cap mats were carefully removed and the wells allowed to drain for 10 mins. The wells are then subjected to a thorough washing procedure (see below) in order to remove unreacted reagents from the resulting resin samples.

Post reaction, the derivatised Sepharose beads undergo a thorough washing procedure consisting of a series of separate wash steps (see below) to ensure all unreacted compounds are removed prior to target screening. All wash steps constituted 5 ml well⁻¹. Wash with 1) 100% MeOH; 2) 50% DMF+50% MeOH (v/v); 3) 50% DMF (v/v in water); 4) water; 5) 0.1 M HCl; 6) water; 7) 0.2M NaOH in 50% IPA; 8) 2× water and 9) 20% EtOH (v/v in water). The washed beads were then stored in 20% EtOH (v/v in sterile deionised water), at 4° C., until required.

To vary the isonitrile component, the same library can be prepared as described above, but using a different isonitrile component at different positions in the reaction block. In this manner, a number of different libraries can easily be generated with different isonitrile components, thus effectively giving rise to a 3D array of ligand structures.

### Library Components

| Number | Structure | Amine |
|---|---|---|
| A1 | 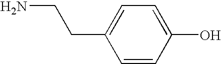 | Tyramine |
| A2 | 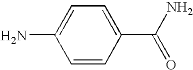 | 4-amino benzamide |
| A3 | 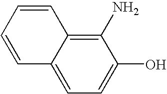 | 1-amino-2-naphthol |
| A4 | 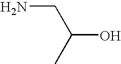 | 1-amino-2-propanol |
| A5 | 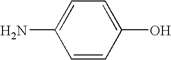 | 4-aminophenol |
| A6 | 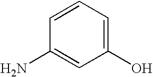 | 3-aminophenol |
| A7 | 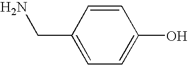 | 4-hydroxybenzylamine |
| A8 | 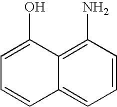 | Amino-8-naphthol |

The table above shows the structure of the amine components of the hIgG-binding Ugi combinatorial library

| Number | Structure | Carboxylic acid |
|---|---|---|
| C1 | 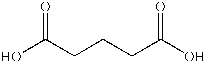 | Glutaric acid^{*} |
| C2 | 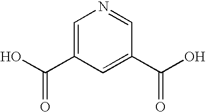 | 3,5-pyridine dicarboxylic acid^{*} |
| C3 | 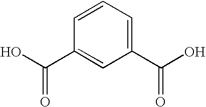 | Isophthalic acid^{*} |
| C4 | 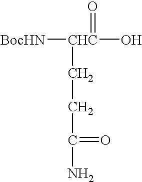 | Boc-Glutamine |
| C5 | 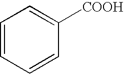 | Benzoic acid |
| C6 | 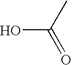 | Acetic acid |
| I1 | 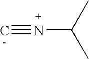 | Isopropyl isocyanide |

The table above shows the structure of carboxylic acid components (C1-C6) and the isonitrile component (I1) of the hIgG-binding Ugi combinatorial library. (Note: isopropyl isocyanide remained conserved for the entire combinatorial library)* The dicarboxylic acid components were first incubated (10 min, R.T.) with equimolar NaOH to protect half of the available COOH groups to avoid cross-linking between adjacent formed scaffold structures on the Sepharose bead. Post reaction washes caused efficient de-protection revealing carboxylic acid groups in the final ligand structure.

### Qualitative Ugi Ligand Fluorescence Studies

Ligands were generated (2.5 g resin scale) using aldehyde-activated Sepharose beads CL-6B (26 μmol g⁻¹ moist weight gel) as described above. For the amine-based pyrene ligand 1-pyrene methylamine, Boc-glycine (carboxylic acid component) and isocyano-cyclohexane (isonitrile component) (all components used 325 μmol (i.e. 5× mol. excess at 2.5 g scale)) dissolved in methanol (5.0 ml), added to the resin and incubated with gentle shaking at 50° C. for 42 h in a 60 ml square-necked Nalgene bottle. The carboxylic acid-based pyrene ligand (B, D) was prepared in the same manner using the amine component 4-aminophenol (A5) and the isonitrile component isocyano-cyclohexane. After incubation, the beads were carefully washed (as described above) and 5.0 μl of a prepared 50% slurry was pipetted onto a microscope slide and viewed using an Olympus CX40 microscope, a Nikon EFD-3 filter (λₑₓ=330-380 nm), a Nikon mercury 100 W lamp and a Kodak DC290 zoom digital camera.

### Chromatographic Screening Protocol and Total Protein Quantitation

The resulting synthesised ligand adsorbents (0.4 ml ligand—50% prepared slurry) were gravity-packed into 4.0 ml (0.8×6 cm) polypropylene columns (200 μl c.v.), prepared for chromatographic analysis (regenerated (0.1 M NaOH, 30% isopropanol, 10 c.v), washed (sterile deionised H₂O, 10 c.v.) and equilibrated (10 mM Na₂HPO₄, 150 mM NaCl, pH 7.4, 10 c.v)) prior to loading (1 c.v, 500 μg ml⁻¹ hIgG/hFab/hFc reconstituted in equilibration buffer). 1 c.v. fractions were collected (10×F.T., 10× elution) and analysed using a standard Bradford assay protocol (Coomassie Plus assay reagent, Pierce, UK) to determine the total protein content in each collected column fraction. This simple target screening methodology will subsequently be referred to as standard chromatographic conditions in the following text.

### Solution-Phase Synthesis

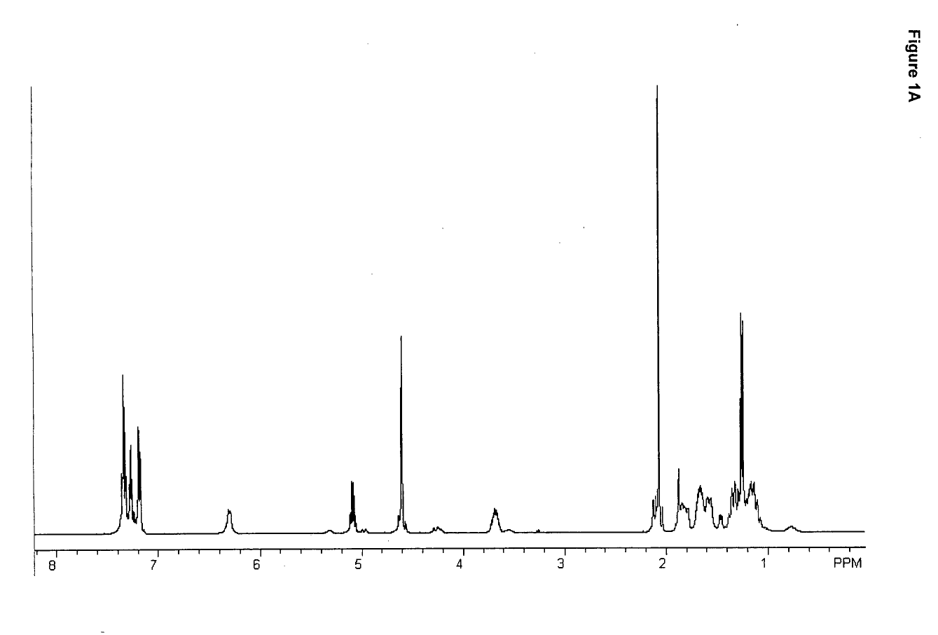

Sepharose beads are susceptible to damage under severe reaction conditions such as high temperature (>100° C.), non-polar solvents and strong mineral acids. Hence mild reaction conditions are considered desirable for library synthesis as well as larger scale-up reactions. To assess the basic kinetics of the Ugi reaction, we used mild reaction conditions (R.T. in methanol) in solution-phase by reacting together acetic acid, benzylamine, acetaldehyde and isocyano-cyclohexane to ensure acceptable product formation. The product 5 was obtained in 68% yield (after recrystallisation from 20% hot ethanol). The identity of the Ugi adduct 5 in was further confirmed by ¹H and ¹³C NMR as shown below in FIGS. 1 (A) and (B) respectively as well as mass spectroscopy (m.p 119-120° C. m/z (EI) 303.41 (M+1, 100%). Found: M+1 303.2074. C₁₈H₂₇N₂O₂ requires 303.207253).

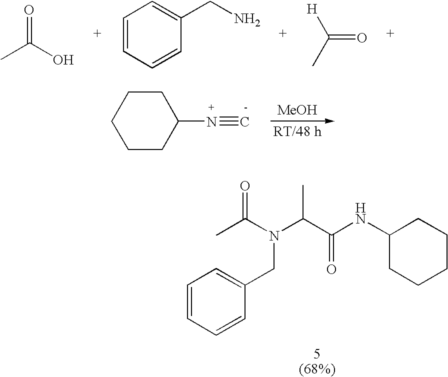

### Evidence of Ugi Scaffold Formation

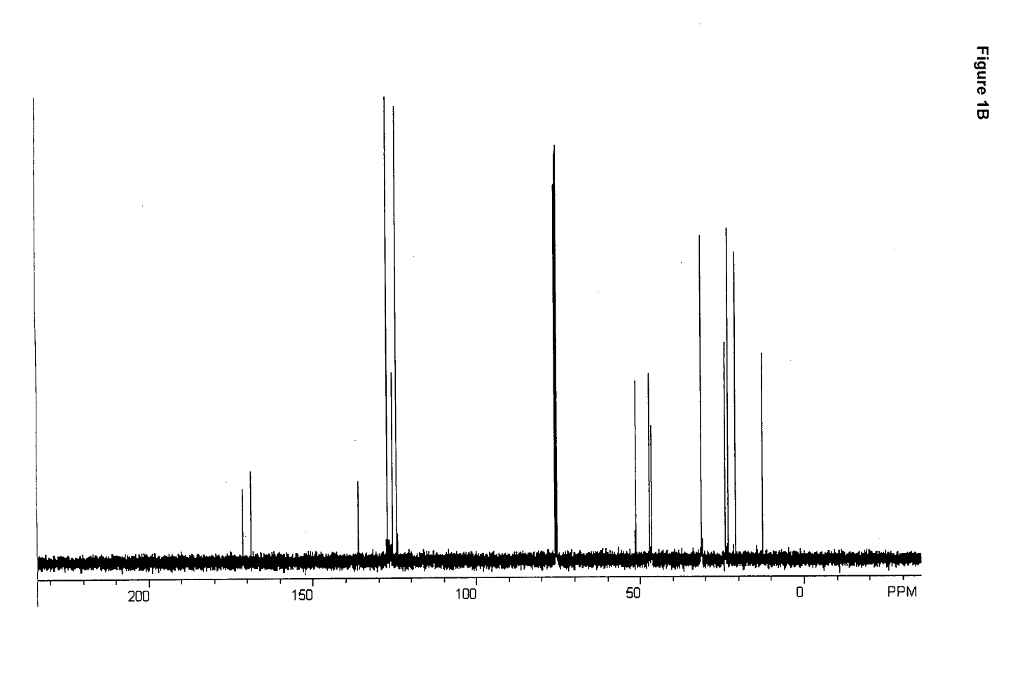

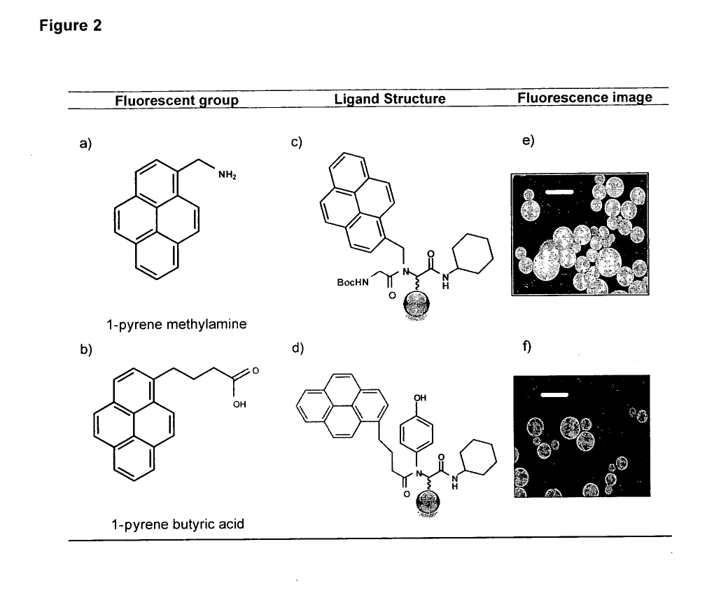

Evidence for Ugi scaffold formation in situ was achieved qualitatively through “on bead” fluorescence studies (FIG. 2). The pyrene-containing amine component (FIG. 2a) and pyrene carboxylic acid component (FIG. 2b) were separately integrated into the Ugi scaffold (FIGS. 2c and d respectively) and subsequently viewed using fluorescence microscopy (FIGS. 2e and f). Integration of the amine-based 1-pyrene methylamine into the Ugi scaffold (structure shown in FIG. 2) provides clear evidence of imine formation with the immobilised aldehyde-activated resin, the first recognised step in the Ugi reaction mechanism. It is thought that the last of the four components to form an integral complex within the Ugi scaffold is the carboxylic acid component and so evidence of carboxylic acid-based 1-pyrene-butyric acid integration also suggested complete formation of the Ugi ligand substituted on the matrix support. Thorough washing also ensured components were not just simply being adsorbed onto the surface of the hydrophilic Sepharose bead and control experiments adding the pyrene components to the aldehyde-activated matrix support also confirmed this point (data not shown).

FIG. 2—Fluorescent ligands used for qualitative evidence of in situ Ugi scaffold formation. a) 1-pyrene methylamine; b) 1-pyrene butyric acid; c) 1-pyrene methylamine integrated into the Ugi scaffold: Boc-glycine, isonitrile: isocyano-cyclohexane); d) 1-pyrene butyric acid integrated into the Ugi scaffold amine: 4-aminophenol, isonitrile: isocyano-cyclohexane); e) Fluorescence image of 1-pyrene methylamine ligand (0.03 sec exposure, ×10 magnification); f) Fluorescence image of 1-pyrene butyric acid ligand (0.25 sec exposure, ×10 magnification). Scale bar (˜100 μm). Fluorescence studies performed using an Olympus CX40 microscope, a Nikon EFD-3 filter (λₑₓ=330-380 nm), a Nikon mercury 100 W lamp and a Kodak DC290 zoom digital camera.

### Rational Library Design

The selection of library components was based on previously described immunoglobulin-binding ligands originally identified at the Institute of Biotechnology, University of Cambridge, UK, together with ligand information obtained from the recent scientific literature.

A number of lead ligands have emerged from various triazine-based combinatorial libraries which have proved successful for both whole and fragmented IgG purification via affinity chromatography. The artificial protein A (ApA) ligand (Li et al., 1998)) eluted hIgG from human plasma to an absolute purity of 98% and showed an apparent binding capacity of 20.0 mg IgG g-1 moist weight gel. This ligand is thought to mimic the continuous Phe132-Tyr133 dipeptide located at the end of a helix within fragment B of the naturally occurring protein A (from Staphylococcus aureus) (SpA). This particular region of the naturally occurring protein is known to bind the CH2 and CH3 domains of IgG predominantly through hydrophobic interactions, hence the ability for ApA to bind IgG at both the conventional Fc binding site and the alternative Fab binding site (Hillson et al., 1993).

In this study, Ugi ligands have been identified that show binding to whole hIgG in addition to specific Fab and Fc binding ligands. The components of this combinatorial library selected to mimic ApA-like interactions include benzoic acid (C5), tyramine (A1), 4-aminophenol (A5), 3-aminophenol (A6) and 4-hydroxybenzylamine (A7).

The triazine-based immunoglobulin specific ligands are shown below. (A) Artificial protein A; (B) optimised IgG-binding ligand 22/8; (C) PpL biomimetic ligand 8/7. Note: The ligand nomenclature used refers to combinatorial triazine library components.

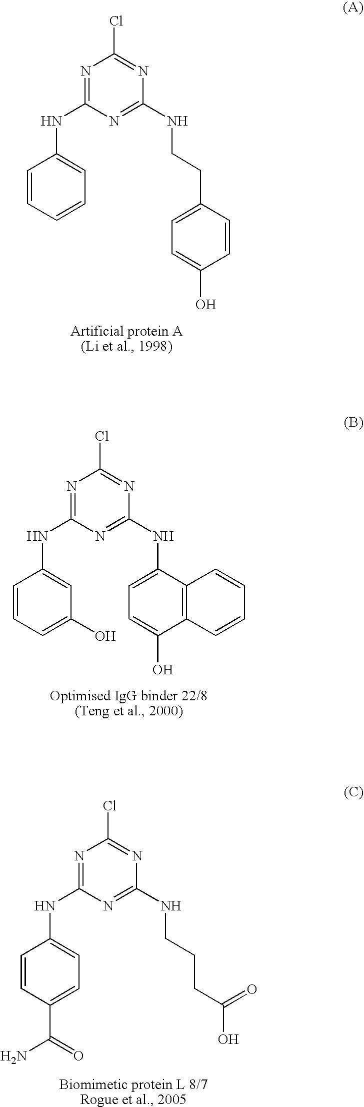

By a gradual process of optimisation over a number of years using an intentionally biased combinatorial library of related ligand structures (Teng et al., 1999), the ApA ligand evolved structurally into the near-neighbour triazine ligand 22/8 (Teng et al., 2000). The hydrophobic ligand 22/8 was shown to elute hIgG with a recovery of 67-69% and a purity of 97-99%, depending on the pH value of the elution buffer used and showed an improved binding capacity of 51.9 mg IgG g⁻¹ moist weight gel, far higher than that of the previous ApA ligand. Furthermore, the ligand 22/8 also showed binding to Fab and Fc fragments in a manner similar to that of ApA and SpA.

The components introduced into this library to mimic interactions displayed by ligand 22/8 included the amines: tyramine (A1), 4-aminophenol (A5), 3-aminophenol (A6) and 4-hydroxybenzylamine (A7) and the naphthol derivatives 1-amino-2-naphthol (A3) and amino-8-naphthol (A8). It is thought that although SpA interacts with the Fab fragment, the governing interaction by which SpA interfaces with IgG is through the Fc region and so the components described above, selected to mimic such an interaction, when incorporated into an Ugi scaffold would be expected to interact in a similar manner and potentially yield Fc-specific ligands. In a further attempt to generate Fab-specific Ugi ligands, a number of separate components were also incorporated into this library that resembled the structure and functionality of the protein L mimetic ligand 8/7 (Roque et 2005b). Protein L (PpL) is a bacterial surface protein (from Peptostreptococcus magnus) with a high affinity towards the light chains of the κ1, κ3 and κ4 subgroups, but not to κ2 and λ subgroups (Nilson et al., 1992; Enokizono et al., 1997) and thus interacts with both whole and light chain-related IgG fragments (i.e. Fab and scFv). Similar functional elements of this particular ligand are reflected in this present Ugi library by the amine component 4-aminobenzamide (A4) together with the carboxylic acid components glutaric acid (C1), 2,4-pyridine dicarboxylic acid (C2), isophthalic acid (C3) and Boc-protected glutamine (C4). Also, seven of the nine putative lead ligands that emerged from the triazine-based library mimicked tyrosine (i.e. contained tyramine (A1)), further justifying this component's inclusion into the Ugi library selection process. Additional supporting evidence for the importance of mimicking the tyrosine group comes from studies describing the 140-fold decrease in affinity that PpL shows for IgG upon chemical modification of the PpL residues Tyr⁵¹ and Tyr⁵³ respectively (Beckingham et al., 2001). Incidentally, the final candidate ligand (8/7) that was chosen did not include the tyrosine functional group due to the higher level of specificity shown by ligand 8/7.

The recent literature suggests that there are seven key residues conserved in different PpL domains and largely buried upon complex formation from the PpL domain (strand β2 and α1 helix) involved in the primary interaction between PpL and IgG light chains. These residues are listed below, followed by their italicised Ugi library analogues: Gln³⁵: 4-aminobenzamide (A2) and Boc-glutamine (C4); Thr³⁶: 1-amino-2-propanol (A4); Ala³⁷: acetic acid (C6); Glu³⁸: glutaric acid (C1), 2,4-pyridine dicarboxylic acid (C2) and isophthalic acid (C3), Phe³⁹: benzoic acid (C5); Lye⁴⁰ and Tyr⁵³: tyramine (A1), 4-aminophenol (A5), 3-aminophenol (A6) and 4-hydroxybenzylamine (A7).

### Ugi Library Screening and Putative Lead Selection

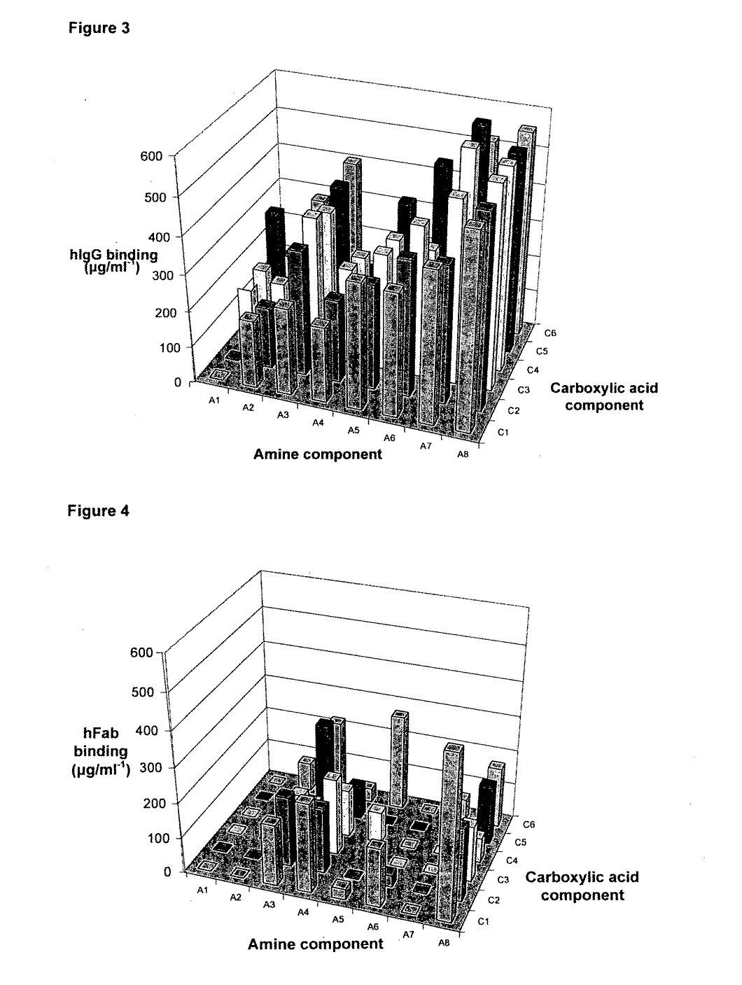

Non-optimised standard chromatographic screening conditions were established to determine the efficacy of emerging library candidates in an attempt to rapidly identify lead candidates for further development and evaluation. Data for the ligand adsorbents is shown in FIGS. 3, 4 and 5 for hIgG, hFab and hFc binding respectively, as determined by a standard Bradford assay (Bradford 1976). Analysis of the data prompted the selection of lead ligands for whole hIgG, and specific hFab and hFc fragment binding ligands.

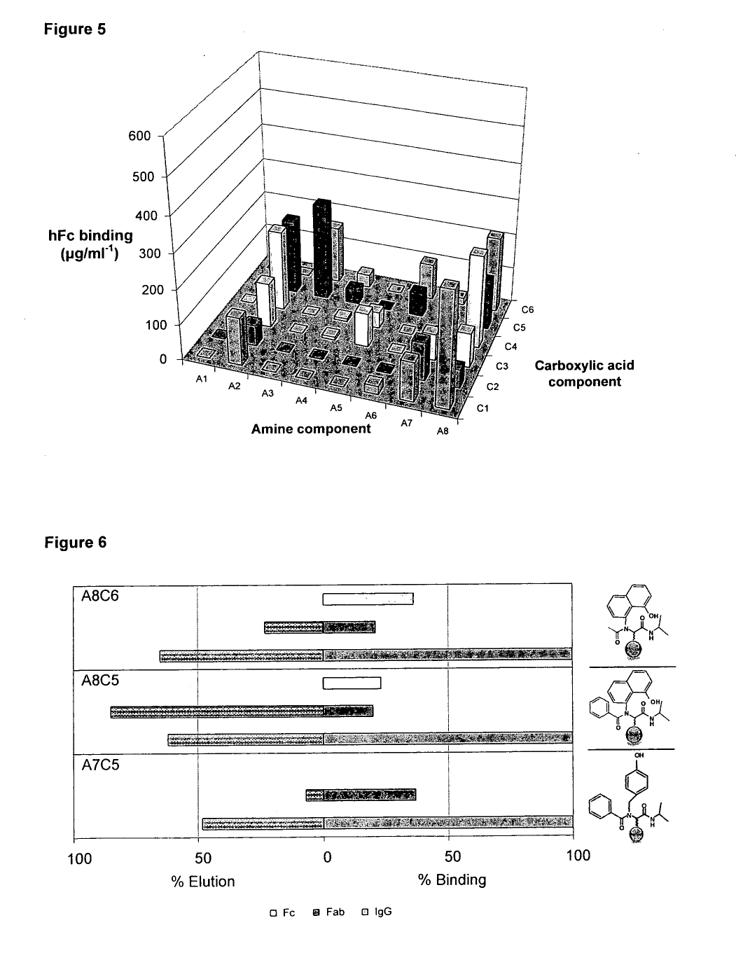

The main criteria for lead ligand selection was potential hIgG-binding based on the observed total hIgG binding capacity achieved. The candidate ligands A7C5, A8C5 and A8C6 showed 100% hIgG binding from an initial 500 μg ml-1 load applied to each column. See FIG. 6 for hIgG lead structures and non-optimised % adsorbtion/desorbtion. Interestingly, the putative lead ligand A7C5 represents a near-neighbour functional mimic of the ApA ligand thus supporting the overall library selection process used. Conversely, the direct ApA mimic present in the Ugi library (A1C5) did not perform as well as A7C5 (43% hIgG binding) possibly due to the additional flexibility contributed by the tyramine component (A1) as compared to the more rigid 4-hydroxybenzylamine component (A7) and may also help to explain the ˜57% loss in the binding capacity observed. From detailed binding analysis of the Ugi library, all ligands containing the amino-8-naphthol component (A8) showed 100% hIgG binding in addition to varied Fab and Fc binding profiles. This suggests that ligands containing the A8 component may exhibit binding properties similar to that of the triazine ligand 22/8 (i.e. immunoglobulin-binding for whole, Fab and Fc fragments).

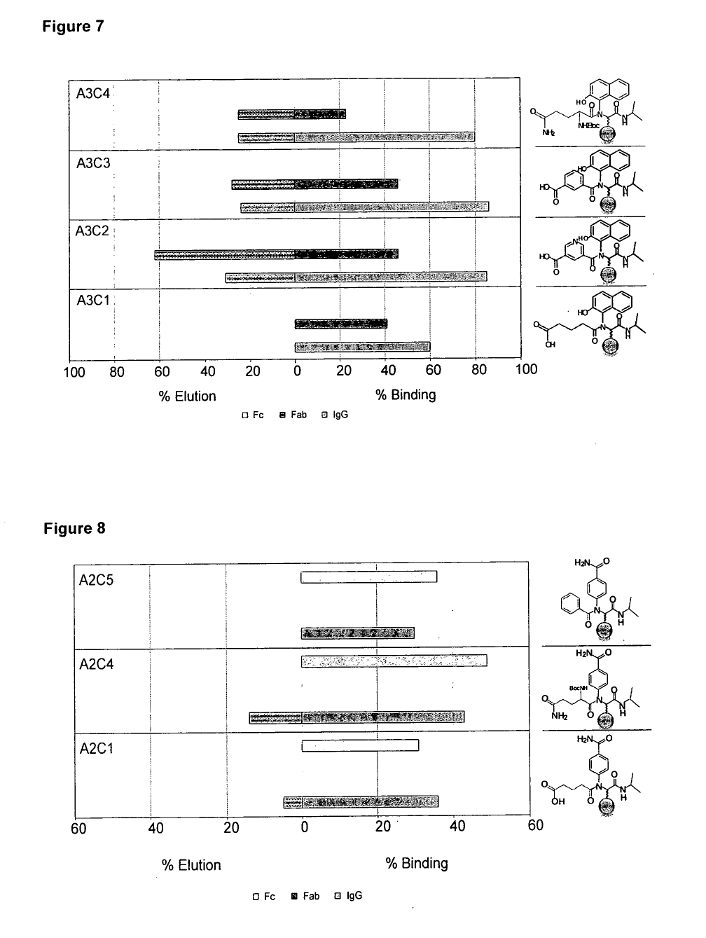

The amino-naphthol component 1-amino-2-naphthol (A3) also displayed promising whole hIgG binding (approximately 60-86% binding strongly dependent on the carboxylic acid component) however, for carboxylic acids C1-C4, complete specificity to the Fab fragment was observed (i.e. 0% Fc binding). This may explain the reduced hIgG binding observed for the A3 component as compared to the A8 component. Based on this observation, A3C1, A3C2, A3C3 and A3C4 were selected as putative Fab leads for further optimisation studies. See FIG. 7b for hFab lead structures and non-optimised % adsorbtion/desorbtion.

The selection of the proposed hFc lead candidate ligands A2C2, A2C4 and A2C5 (FIG. 8) also provided some evidence that the Ugi and triazine scaffolds do differ in terms of ligand-binding behaviour. The ligand A2C1 is a direct equivalent to the triazine-based biomimetic protein L 8/7 in terms of substituted functional groups however when the same functionalities are substituted on the Ugi scaffold, this ligand apparently shows a complete specificity for the Fc fragment. We also observed that six out of the seven whole IgG and Fab-specific leads include one of the two closely-related naphthol components (A3 or A8) and the majority of these ligands responded well to the non-optimised elution conditions (0.1M NaHCO3, 10% (v/v) ethylene glycol, pH 10.0). Conversely, none of the A2-related hFc leads responded well to the chosen elution conditions strongly suggesting that the mode of binding to hFc differs for these ligands from that of whole IgG and specific hFab leads identified in this study. This is possibly due to the nature of solvent-exposed residues present in the vicinity of the binding site, this in turn constitutes the type of interaction that can occur between these ligands and their respective targets. The overall hydrophobicity of two randomly selected hFab and hFc fragments (PDB codes 1AQK and 1H3W respectively) were compared using a computational method as part of a software package, HyperChem 7.5 Professional (http://www.hyper.com/index.htm). LogP values for the two protein fragments (hFab log₁₀ P=−1573.3; hFc log₁₀ P=−510.4), based on solvent-exposed amino acid residues, suggested that hFc is considerably more hydrophobic than that of hFab fragment (Fc>Fab 3× approx.). This type of analysis may also help to define the final optimised adsorbtion and desorbtion conditions required for the selected Fab and Fc lead ligands for large-scale purification processes.

The binding capacities reported above were determined with a single pass of the target protein incorporating an average ˜30s column residency time with non-optimised adsorbtion/desorbtion conditions. The aim of this simple screening procedure was to determine a relative binding capacity value for every ligand in order to simplify the lead selection process. It is further envisaged that accurate frontal analysis-derived binding capacities will also be required to determine lead ligand candidates (1 ml c.v scale) under optimised conditions to reveal comparable values to currently available IgG-binding ligands. These ligands typically display binding capacities in the range of ˜40 mg ml-1 gel moist weight. Recently, other suitable potential candidate ligands have also emerged from this library for complete and fragmented immunoglobulin targets. Initial lead selections were primarily based on absolute binding capacity, specificity and response to the non-optimised absorption and desorbtion conditions applied during the screening procedure. It is also envisaged that these lead candidates will be further optimised and characterised through the introduction of variable-length spacer arms (C2-C8), further optimisation of the chromatographic conditions used and the utilisation of variable isonitrile components to possibly improve upon ligand binding and elution behaviour. However, other candidate ligands may also be considered if required, taking advantage of this iterative approach to ligand design.

The data presented here also revealed specific families of amine components, substituted onto the Ugi scaffold, which provided specificity to hFab (A3 and A4) and hFc (A2 and A7) fragments and therefore it is not surprising all the identified hFc leads contain the A2 amine component. In addition, the A8 amine component produced a number of non-specific, relatively high-capacity adsorbents for binding to both whole IgG and fragmented targets thus justifying its inclusion in two of the whole IgG leads. Conversely, the trend identified for the incorporated carboxylic acid components was not so readily identifiable which may suggest that the amine component is of primary importance in determining the ligand-target binding interface.

### Identification of Affinity Ligands for Factor VIII

In a further study, a collection of compounds was prepared to identify possible affinity ligands for Factor VIII. Each compound was prepared in a manner similar to that described for the IgG experiments above. Thus, an aldehyde functionalized linker attached to a support was reacted with a carboxylic acid, an amine and an isonitrile in an Ugi multicomponent reaction. The products were then screened for their ability to bind Factor VIII. The binding ability of each compound was compared against previously identified ligands.

This project investigated the possibility of developing small molecule affinity ligands to improve upon the cost-effectiveness of large-scale purification of a full-length recombinant Factor VIII. This product is currently used as a proven clinical biotherapeutic molecule in the treatment of Haemophilia A and related blood disorders.

The advantage of small molecule ligands over the existing C7F7 monoclonal antibody resin approach (such as that used by Bayer) is that small molecule ligands are significantly cheaper to produce and can withstand harsher resin regeneration conditions for multiple column runs which at present can not be used for the Bayer antibody column.

A series of affinity ligands were developed based on the incorporation of specific functional groups onto a generic Ugi scaffold which itself was substituted through an aldehyde moiety established on a solid phase matrix support (Sepharose CL-6B) in a separate procedure. The underlying chemistry representing the formation of the final ligand structure on the Sepharose bead as a single multicomponent reaction requires the use of four separate components an aldehyde (R1), primary/secondary amine (R2), isonitrile (R3) and carboxylic acid (R4).

### Linker and Support Preparation

The linker and support used were the same as those described above in relation to the IgG experiments.

### Preparation of the Compound Collection

A collection of compounds was prepared following the Ugi-based protocol described above in relation to the IgG experiments.

The compounds prepared have the general structure given below:

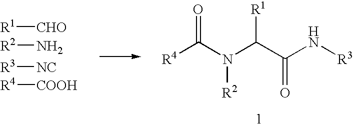

Where R¹ is the linker and support, R² is the amine component, R³ is the isonitrile component and R⁴ is the carboxylic acid component.

The combinations of carboxylic acid, isonitrile and amine component used to generate the collection are given below:

| | Acid | Amine | Isonitrile | Factor VIII |
|---|---|---|---|---|
| Name | Component | Component | Component | Bound (μg/mL) |
| U1 | 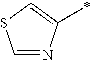 | 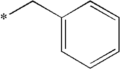 | 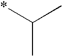 | 63.1 |
| U2 | | 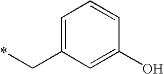 | | 71.9 |
| U3 | | 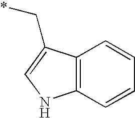 | | 85.8 |
| U4 | | 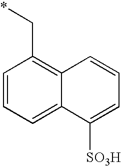 | | 98.9 |

Each compound, U1 to U4, was screened by Factor VIII microplate assay, and the results are given in the table above. These results show that increasing aromatic heterocyclic complexity improves Factor VIII binding.

The results compare favourably with the binding capability of triazine ligand 34/43 (106.3 μg/mL) that has been previously prepared by the present inventors and is a known ligand for factor VIII.

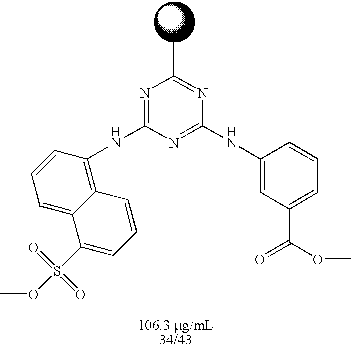

The results of the initial Factor VIII microplate assay show that as the amine component is varied in terms of increasing structural and electrostatic complexity there is also an observed increase in Factor VIII binding which is comparable to similar increases seen for triazine-based lead ligands. This suggests that although the two scaffolds (triazine and Ugi) differ greatly in terms of chemical structure, the influence of individual functional groups on Factor VIII binding is retained thus allowing for a similar process of lead discovery to take place.

On the basis of the results obtained from the initial collection, additional ligands were prepared and screened in relation to Factor VIII binding and elution behaviour. These studies have been performed either using a simple microtitre plate assay (resin volume 40 μL/well) to determine approximate Factor VIII binding affinity followed by more detailed studies using gravity-flow packed resin columns (0.5 mL resin volume). These packed column studies are more accurate and allow simple experiments to be conducted to determine absolute resin affinity by gradual saturation of the column (200 μL, 100 μmL) and following the protein concentration in the eluate after serial addition of each factor VIII aliquot applied to the column. In this way, the binding can be established and elution behaviour of each ligand in parallel.

Another type of study used to investigate the elution behaviour of selected ligands is to initially attempt to saturate a 0.5 mL packed column by repeated addition (×10) of a single Factor VIII aliquot (1.2 mL 100 μg/mL) after which the protein concentration can be empirically determined followed by subsequent addition of a series of wash and elution buffer aliquots (400 μL). Therefore the binding and elution behaviour of the ligand can be followed under conditions of high initial Factor VIII load. A feature of these studies has led to the observation that the selected Ugi ligands respond well to high concentrations of monovalent and divalent cation salts (CaCl₂, NaCl) with respect to Factor VIII elution. It is suggested in this report that this may form the basis for a differential purification approach for Factor VIII. It may also be possible to remove significant levels of background host cell protein binding by correct identification of binding, wash and elution conditions.

An additional library was then prepared using the Ugi multicomponent reaction. The acid, amine and isonitrile components are given in the table below along with the Factor VIII microplate assay data. The aldehyde component was again an aldehyde-functionalized sepharose.

| | Acid | Amine | Isonitrile | Factor VIII |
|---|---|---|---|---|
| Name | Component | Component | Component | Bound (μg/mL) |
| 5U | 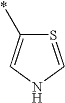 | 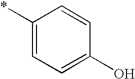 | 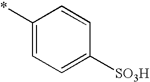 | 97.8 |
| 6U | 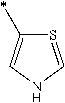 | 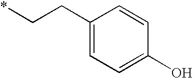 | 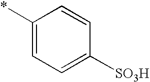 | 66.1 |
| 7U | 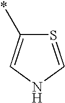 | 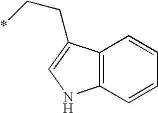 | 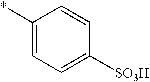 | 104.8 |
| 8U | 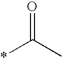 | 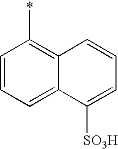 | 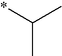 | 110.1 |
| 9U | 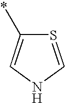 | 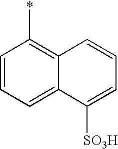 | 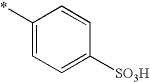 | 107.5 |
| 4U | 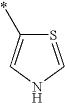 | 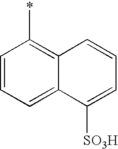 | 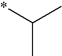 | 109.6 |

Ugi ligands (4-9U) and triazine ligand 34/43 were initially screened by Factor VIII microplate assay the results show a general trend towards a bicyclic aromatic ring in the amine (R2) position having a positive influence on Factor VIII binding. The presence of an additional sulfanilic acid moiety in the isonitrile (R3) position did not appear to strongly influence Factor VIII binding similarly the presence of the thiazole moiety in the carboxylic acid (R1) position did not provide a strong additional binding potential. It appeared that the original triazine ligand 34/43 still possessed the strongest Factor VIII binding and elution characteristics based on these studies.

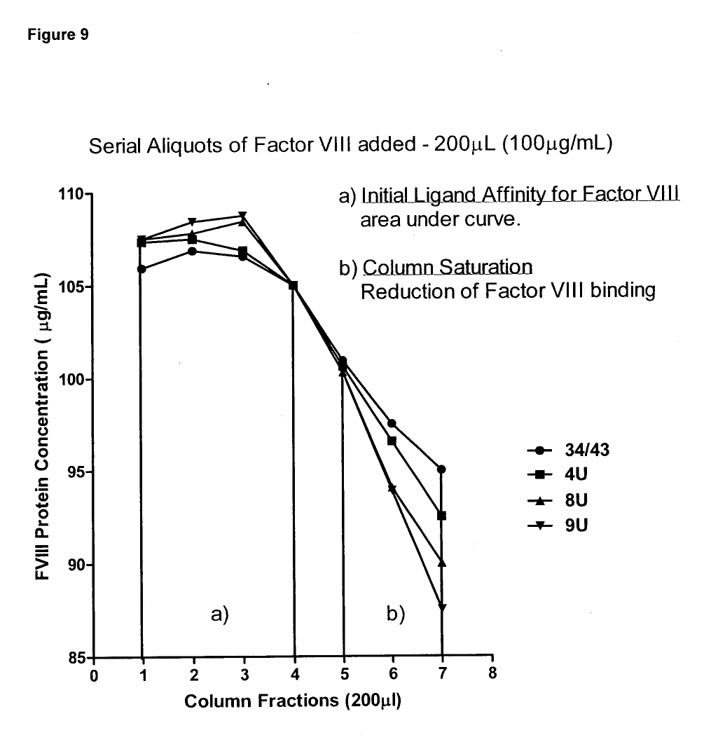

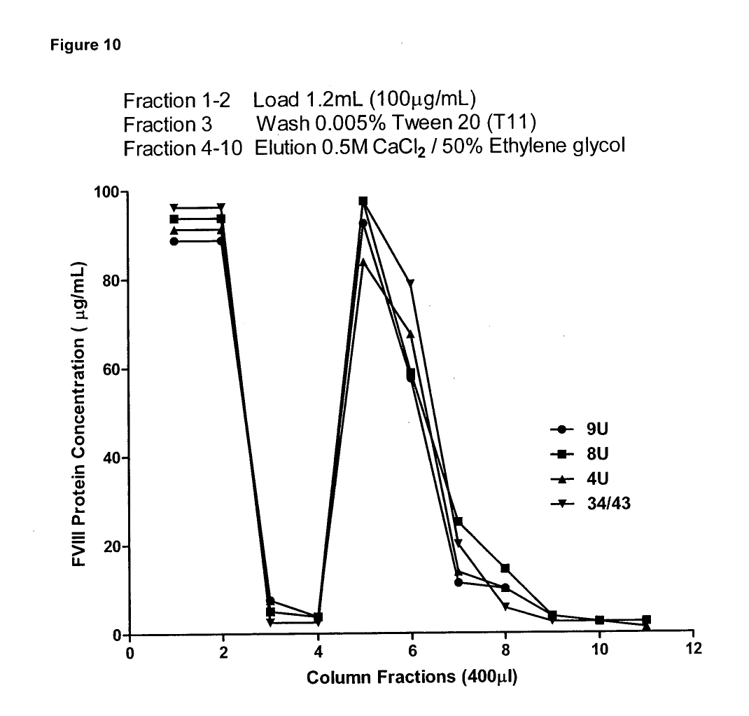

Further investigation of ligands selected from this series (4, 8, 9U+ligand 34/43) using packed columns (0.5 mL ligand resin) identified that the Factor VIII binding potential of 4U, 8U, 9U was significantly lower than that of the triazine ligand 34/43 however the elution behaviour seemed to be comparable to this ligand using similar elution conditions: 0.5M CaCl₂/50% Ethylene glycol/20 Tris.HCl pH 7.0 (see FIGS. 9 and 10).

The reduced Factor VIII binding potential for selected Ugi ligands 4U, 8U and 9U prompted us to design and synthesise additional ligands and suitable control ligands to further investigate the nature of this effect. The current Ugi ligand set is 10-17U which are currently being investigated with respect Factor VIII binding and elution behaviour (See FIG. 7). We initially screened a number of these new ligands and previous examples mentioned in this report by Factor VIII microplate assay. Ligands U14 and U17 contain a nitro and dinitro-benzene moiety to consider a replacement group for benzoic acid used previously and attempt to redefine the ligand structure to take into account the potential binding mode of factor VIII for phosphatidylserine. In this respect, it is clear that we also need to produce a further ligand with benzoic acid substituted at the carboxylic acid (R1) position to compare the potential binding mode with ligands produced so far.

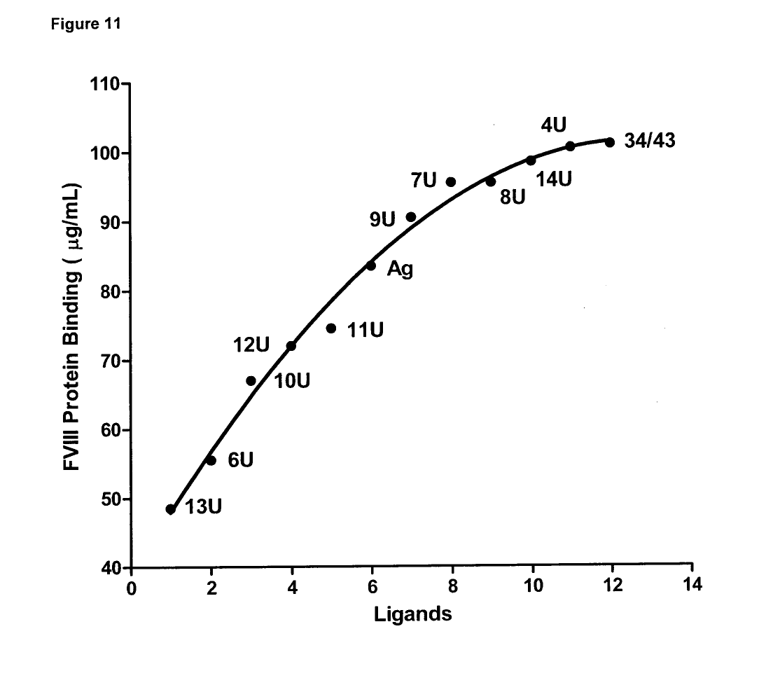

The results of this study showed that the triazine ligand 34/43 consistently produced one of the highest Factor VIII binding potential however it was noted that Ugi ligands 4U, 7U, 8U, 9U and U14 produced similarly high levels of Factor VIII binding. It also appeared that Ugi ligands U10-U13 produced significantly lower Factor VIII binding as judged by this simple assay. It is suggested by these results that the Ugi scaffold itself is not making a particularly strong impact on Factor VIII binding even in the presence of the functional naphthalene moiety (Ugi ligand—U12). It also appears that the combination of suitable spacing provided by the Ugi scaffold from the bead surface and the presence of the naphthalene sulfonate moiety provides a strong Factor VIII binding potential since ligand U10 exhibits a reduced binding potential (See FIG. 11).

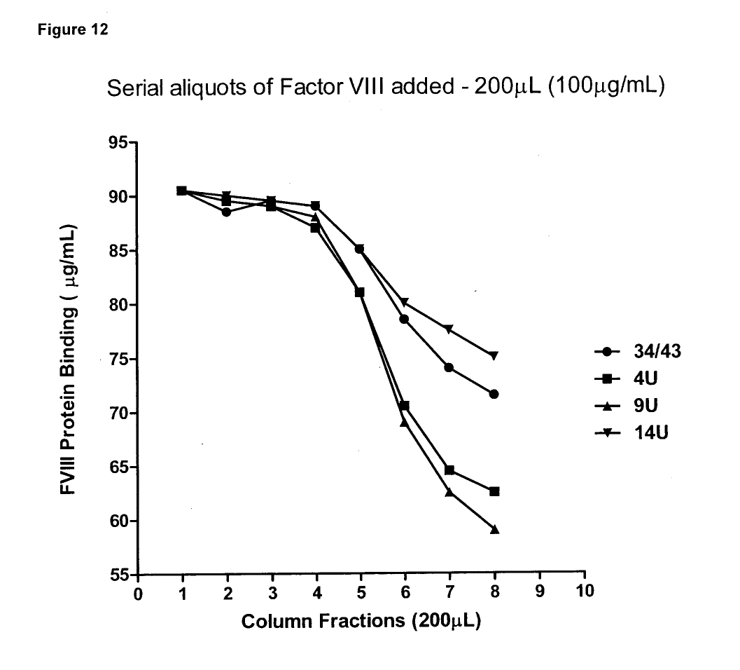

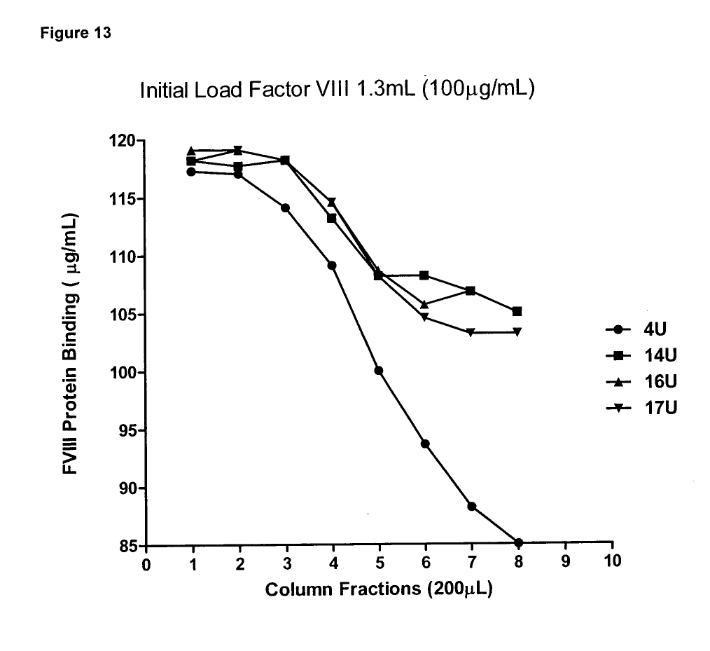

It was felt that the microplate assay did not provide the accuracy required to confirm these results therefore further Factor VIII binding experiments were performed by applying serial Factor VIII aliquots (2004. 100 μg/mL) to 05 mL packed columns to selected Ugi ligands from this set including the triazine ligand 34/43 (See FIG. 12). It was noted from this study that Ugi ligand 14U appeared to show a Factor VIII binding potential higher than that of the triazine ligand 34/43. It also appeared that Ugi ligands 4U and 9U showed a similar reduced Factor VIII binding potential (See FIG. 12). This result has also been confirmed in a separate study of four selected Ugi ligands 4U, 14U, 16U, 17U which suggests that differential Factor VIII binding modes can be identified by suitable ligand design (See FIG. 13). This result strongly suggests that the presence of the benzene aromatic ring at the carboxylic acid (R1) position contributes positively to the overall Factor VIII binding potential. Clearly this needs to be investigated further by the suitable addition of different functional groups which we are currently investigating. Similarly, it is also noticeable that the design of specific inhibitors of Factor VIII directed towards the C2 domain possess a general scheme of four 6C-5C-5C-6C aromatic ring structures and additional electronegative substituents (CF₃, NO₂, ═S, ═O and dichlorobenzene) (Spiegel et al., 2004)

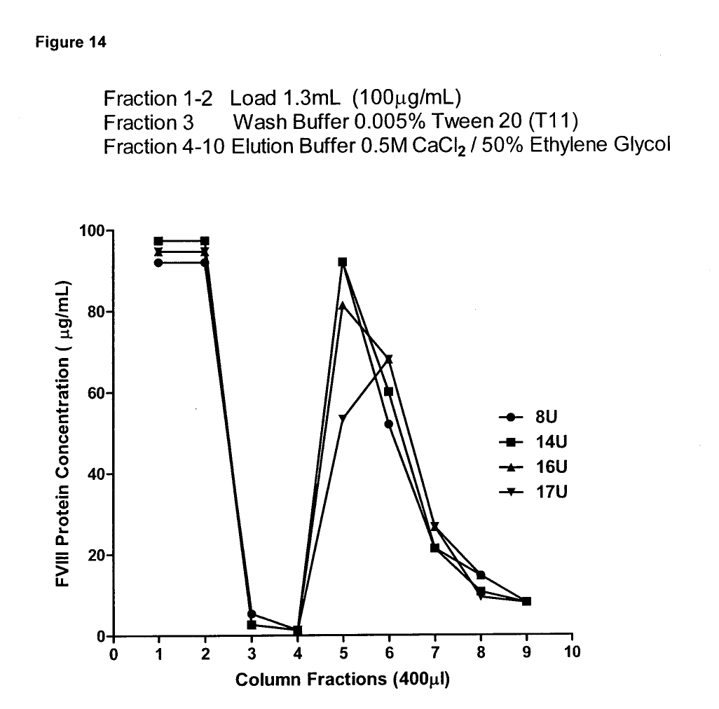

The selected Ugi ligands (U8, U14, U16, U17) were screened for their ability to bind and elute Factor VIII in 0.5 mL packed columns (see FIG. 14). The results show that ligands 8U and 14U behave similarly and elute sharply in the initial fractions with high efficiency. Whereas ligands U16 and U17 appear to elute less efficiently across a broader front. It is suggested that ligand U17 in particular appears to possess a slightly different binding mode to the other ligands which implies that the dinitrobenzene group is strongly influencing either or elution or both.

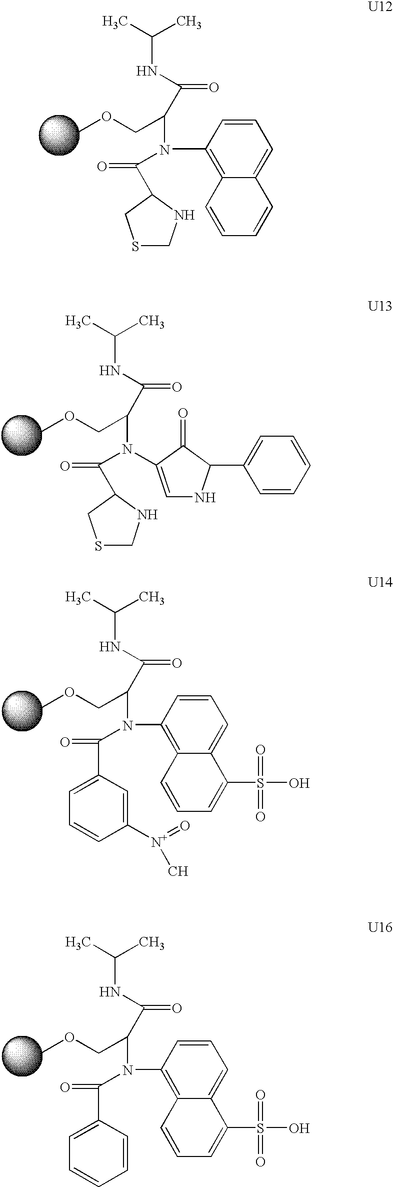

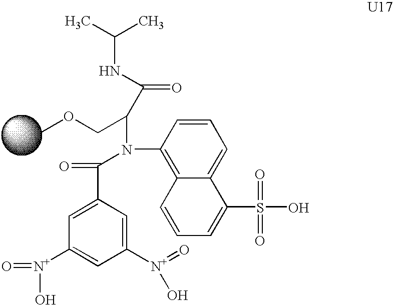

Three control ligands were also prepared:

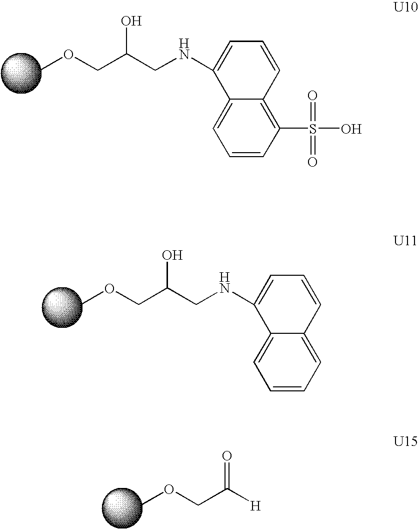

### Modeling Ugi Ligands to Factor VIII-C2 Domain Using the Molegro Virtual Docker Software

A set of training virtual ligands was initially used to identify potential binding modes to two discrete regions of the Factor VIII-C2 domain (See FIG. 12). These two surface cavities differed in size from 10 to 17 Angstroms in radius (See FIG. 13) and were identified in previous studies as potential regions in which suitable ligands may interact favourably with the C2 domain (data not shown). The automated docking software Moldock as part of the Molegro software package (SIM biosystems, USA) was used to assess ligand binding using a total set of 50 random ligand conformations, 4000 separate docking iterations and averaged over three independent programs runs. The program delivers a set of the five best poses (docking modes) found combined with a report of the Moldock score, affinity and other relevant parameters such as reRank Score, total electrostatic energy and total H-bond energy etc. The quantitative data provided by this program is provided as an attached excel file.

The results show that there is some evidence for improved docking modes associated with Ugi ligands 14U, 17U, 20U and 21U associated with either the Moldock score or Affinity parameters in both cavity 1 and 2. The proposed best docking mode is shown for ligand 14U as a surface view (bottom left) and side “stick” view (bottom right) (See FIG. 13). The residues most likely to interact with this ligand are shown as follows Arginine 2220 (blue—positive charge), Glutamine 2316 (lime green—positive/negative charge), Phenylalanine 2196 (red—hydrophobic), Aspaginine 2224 (pea green—positive/negative charge), Valine 2223 (magenta—nonpolar), Histidine 2315 (salmon pink—positive charge). Similarly, the best docking mode is shown for ligand 17U, the residues most likely to interact with this ligand are labelled as mentioned above (See FIG. 14). A noticeable feature of these studies is the potential interaction of surface-exposed arginine residues with either the sulfonate moiety or the naphthalene aromatic ring structure in both cavity 1 and cavity 2. I also include automated docking data for ligands 4U and 34/43 (Bayer code—Ligand 3A) in the excel file which suggests a high-binding potential for cavity 1 for ligand 34/43 as compared to ligand 4U which is in close agreement with the experimental data obtained so far. The docking mode for ligand 34/43 to cavity is particularly good suggesting a high-binding potential to the bottom of the C2 domain structure between the individual finger regions.

The virtual ligand training set is shown below:

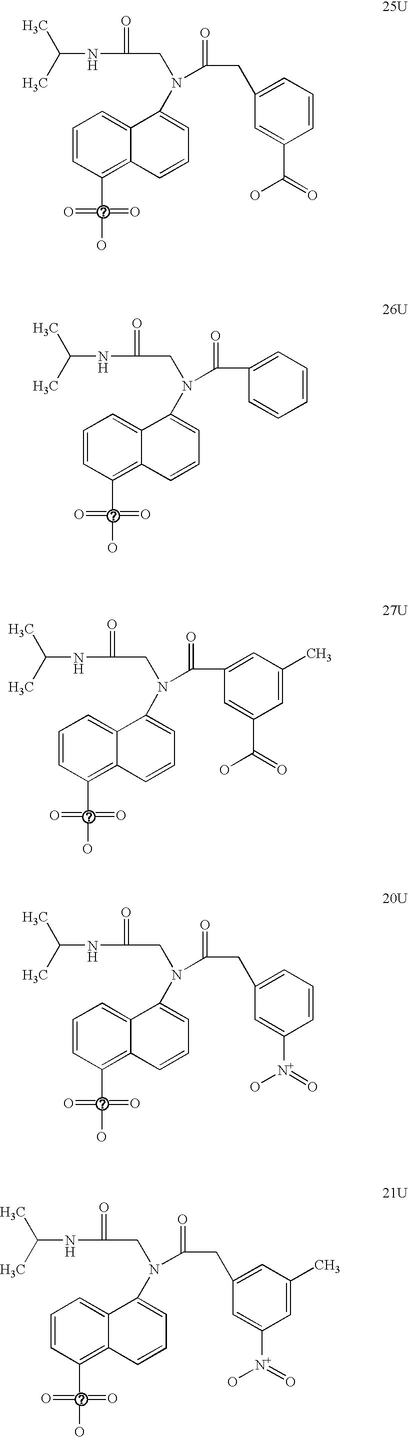

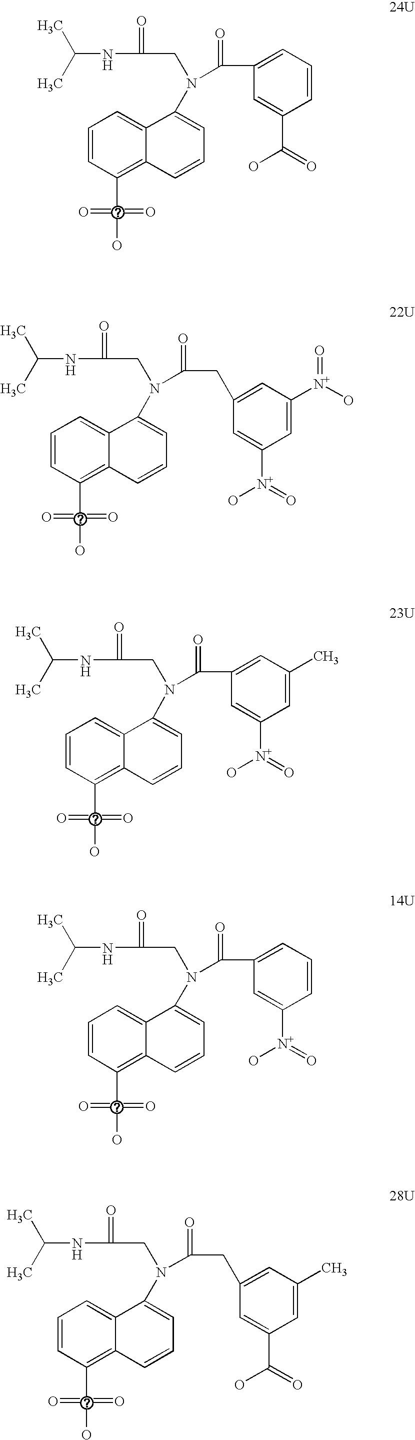

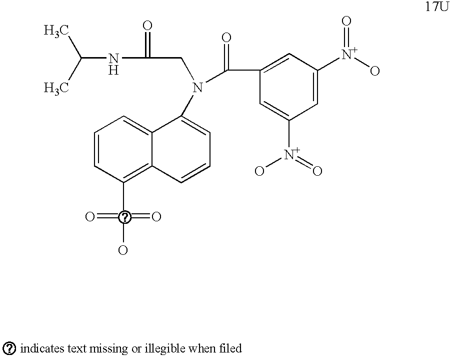

It is noticeable that there are a number of surface-exposed Arginine residues in close proximity to Tryptophan residues in the Factor VIII C1/C2 domains this would allow for the formation of strong Cation-π interactions. It is suggested that one feature of the favourable interaction between the naphthalene sulfonate moiety and the C2 domain may involve such interactions. Friess and Zenobi, 2001 identified positive interactions between Arginine residues in selected proteins and naphthalene sulfonate derivatives by MALDI Mass Spectrometry. It is also known that hot spots involved in protein-protein interactions are enriched in certain residues namely Tryptophan, Tyrosine and Arginine presumably also involved in forming particularly strong interactions (Bogan and Thorn, 1998). The unique arrangement of tryptophan and arginine residues at the distal end of the C2 domain may form interactions with both the VWF protein and phospholipid membranes largely by electrostatic bonds created by positively charged arginine residues and the extended π-cloud produced by tryptophan residues. I am currently investigating the role of the sulfonate moiety with respect to protein binding by surface-exposed arginine residues. In this respect Factor VIII-C2 domain possesses two sulfate-binding sites which are also shared by a number of other proteins. It is known that sulphate and phosphate-binding sites in proteins differ in terms of the residues involved however the Arginine residue appears to be predominantly involved.

### REFERENCES

The following documents are referred to in the description text. Each of these is incorporated herein in its entirety.
Beckingham, J. A., Housden, N. G., Muir, M., Bottomley, S. P. and Gore, M. G. (2001). “Studies on a single immunoglobulin-binding domain of protein L from Peptostreptococcus magnus: the role of tyrosine-53 in the reaction with human IgG.” Journal of Biochemistry 353: 395-401.
Bradford, M. M. (1976). “A rapid and sensitive method for the quantitation of microgram quantities of protein utilizing the principle of protein-dye binding.” Anal. Biochem. 72: 248-54.
Enokizono, J., Wikstrom, M., Sjobring, U., Bjorck, L., Forsen, S., Arata, Y., Kato, K. and Shimada, I. (1997). “NMR analysis of the interaction between protein L and Ig light chains.” J. Mol. Biol. 270(1): 8-13.
Hillson, J., Karr, N., Oppliger, I., Mannik, M. and Sasso, E. (1993). “The structural basis of germline-encoded VH3 immunoglobulin binding to staphylococcal protein A” J. Exp. Med. 178(1): 331-336.
Holliger, P. and Hudson, P. J. (2005). “Engineered antibody fragments and the rise of single domains.” Nature Biotechnology 23(9): 1126-36.
Li, R. X., Dowd, V., Stewart, D. J., Burton, S. J. and Lowe, C. R. (1998). “Design, synthesis, and application of a Protein A mimetic.” Nature Biotechnology 16(2): 190-195.
Lowe, C. R. (2001). “Combinatorial approaches to affinity chromatography.” Current Opinion in Chemical Biology 5(3): 248-256.
Nilson, B. H. K., Solomon, A. and Akerstrom, B. (1992). “Protein L from Peptostreptococcus magnus binds to the k light chain variable domain.” The Journal of Biological Chemistry 267(4): 2234-2239.
Roque, A. C. A., Lowe, C. R. and Taipa, A. M. (2005a). “An artificial protein L for the purification of immunoglobulins and Fab fragments by affinity chromatography.” Journal of Chromatography A 1064: 157-167.
Roque, A. C. A., Lowe, C. R. and Taipa, A. M. (2005b). “Synthesis and screening of a rationally designed combinatorial library of affinity ligands mimicking protein L from Peptostreptococcus magnus.” Journal of Molecular Recognition 18(3): 213-224.
Teng, S. F., Sproule, K., Husain, A. and Lowe, C. R. (2000). “Affinity chromatography on immobilized “biomimetic” ligands synthesis, immobilization and chromatographic assessment of an immunoglobulin G-binding ligand.” Journal of Chromatography B 740(1): 1-15.
Teng, S. F., Sproule, K., Hussain, A. and Lowe, C. R. (1999). “A strategy for the generation of biomimetic ligands for affinity chromatography. Combinatorial synthesis and biological evaluation of an IgG binding ligand.” Journal of Molecular Recognition 12(1): 67-75.
Ugi, I., Meyr, R., Fetzer, U. and Steinbruckner (1959). “Versuche mit Isonitrilen.” Angewandte Chemie 71: 386.
Bogan A A. And Thorn K S. 1998. —Anatomy of hot spots in protein interfaces J. Mol. Biol. 280:1-9.
Friess S D. and Zenobi R. 2001. —Protein structure information from mass spectrometry? Selective titration of arginine residues by sulfonates. J. Am. Soc. Mass Spectrom. 12:810-818.
Spiegel P C. et al. 2004. —Disruption of protein-membrane binding and identification of small-molecule inhibitors of coagulation factor VIII. Chemistry and Biology. 11:1413-1422.
Thomsen R. and Christensen M H. 2006. —Moldock: A new technique for high-accuracy molecular docking. J. Med. Chem. 49:3315-3321.
Novabiochem Catalog 2006/2007, Merck Biosciences Ltd.

1. A collection of compounds wherein each member of the collection is independently a compound according to formula (I) or formula (II): 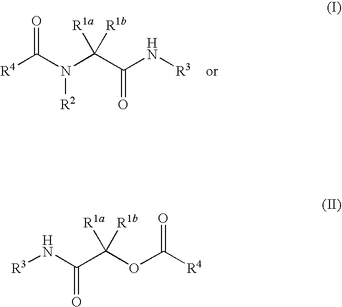
wherein the collection comprises compounds of formula (I) only, compounds of formula (II) only, or a mixture of compounds of formula (I) and (II), and
for compounds of formula (I)
one of R^{1a}, R^{1b}, R², R³ and R⁴ is a group comprising a linker attached to a support,
and the others of R^{1a}, R^{1b}, R², R³ and R⁴ are independently selected from optionally substituted C₁₋₂₀ alkyl, optionally substituted C₃₋₂₀ heterocyclyl or optionally substituted C₅₋₂₀ aryl, and R^{1a}, R^{1b} and R² are additionally selected from hydrogen, and R² is additionally further selected from —S(═O)R⁵ and —C(═S)NR⁶R⁷, wherein R⁵, R⁶ and R⁷ are independently optionally substituted C₁₋₂₀ alkyl, optionally substituted C₃₋₂₀ heterocyclyl or optionally substituted C₅₋₂₀ aryl,
or, optionally, two or more of the others of R^{1a}, R^{1b}, R², R³ and R⁴, together with the atoms to which they are bound, may form a ring; and
for compounds of formula (II)
one of R^{1a}, R^{1b}, R³ and R⁴ is a group comprising a linker attached to a support,
and the others of R^{1a}, R^{1b}, R³ and R⁴ are independently selected optionally substituted C₁₋₂₀ alkyl, optionally substituted C₃₋₂₀ heterocyclyl or optionally substituted C₅₋₂₀ aryl, and R^{1a}, and R^{1b} are additionally selected from hydrogen,
or, optionally, two or more of the others of R^{1a}, R^{1b}, R³ and R⁴, together with the atoms to which they are bound, may form a ring.

2. The collection according to claim 1, wherein R^{1a} is a substituent comprising a linker attached to a support.

3. The collection according to claim 1, wherein R^{1b} is hydrogen.

4. The collection of claim 1, wherein the optionally substituted C₁₋₂₀ alkyl, optionally substituted C₃₋₂₀ heterocyclyl or optionally substituted C₅₋₂₀ aryl may be substituted with one or more substituents independently selected from the group consisting of: acetal, alkyl, aryl, hemiacetal, alkoxy, ketal, hemiketal, heterocyclyl, oxo, thione, imino, formyl, halo, hydroxy, thiocarboxy, thiolocarboxy, imidic acid, hydroxyamic acid, thionocarboxy, ether, nitro, cyano, ether, nitro, nitroso, azido, cyanato, isocyanto, thiocyano, isothioctano, cyano, acyl, carboxy, ester, amido, amino, guanidino, tetrazoyl, imino, amidine, acylamido, ureido, acyloxy, thiol, disulfide, thioether, sulfoxide, sulfonyl, thioamido, sulfinyloxy, sulfate, sulfonamido, sulfonate, sulfamino, phosphino, phospho, phosphinyl, phosphonic acid, phosphonate, phosphate, phosphoric acid, phosphorous acid, phosphoramidite, phosphoramidate, silyl, oxysilyl, siloxy, oxysiloxy and sulfonamino.

5. The collection according to claim 4, wherein the optionally substituted C₁₋₂₀ alkyl, optionally substituted C₃₋₂₀ heterocyclyl or optionally substituted C₅₋₂₀ aryl may be substituted with one or more substituents independently selected from the group consisting of: hydroxy, alkyl, aryl, heterocyclyl, halo, nitro, sulfonic acid, sulfonamido, oxo, thione, carboxy, amino, boronic acid, amido and thioamido.

6. The collection according to claim 1, wherein R² is selected from the list of substituents below:
R² 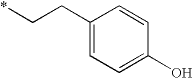 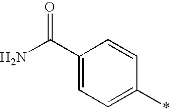 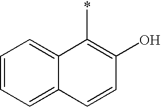 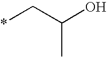 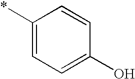 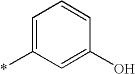 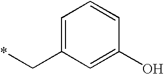 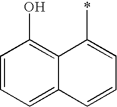 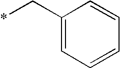 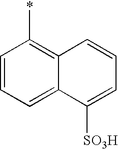 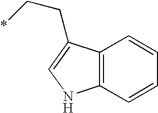 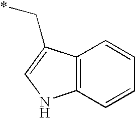 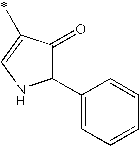 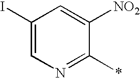 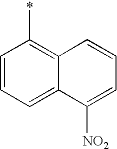 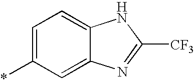 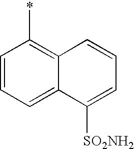 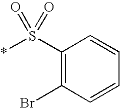 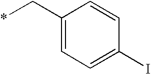 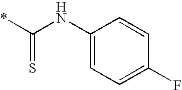 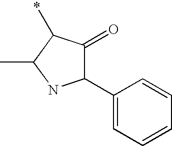 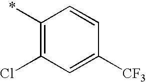 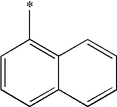 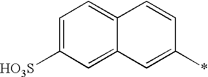 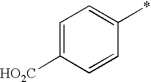 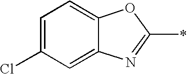 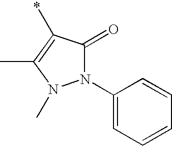 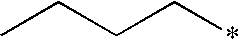 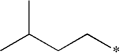 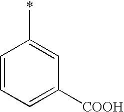 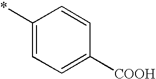 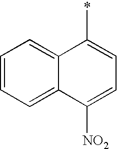 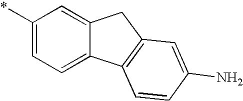 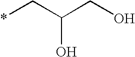 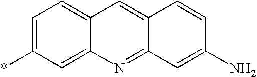 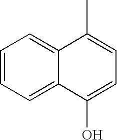 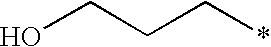 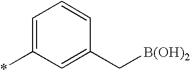 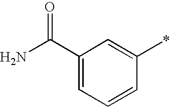 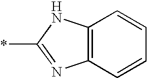 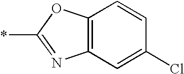 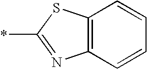 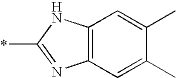 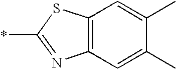 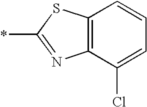 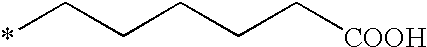 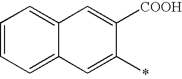 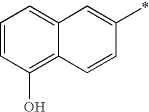 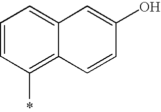 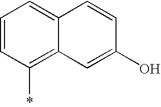 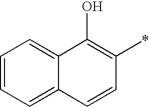 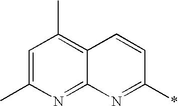 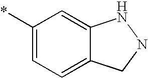 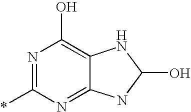 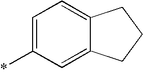 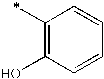 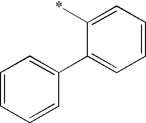 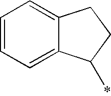 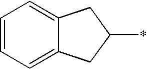 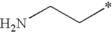 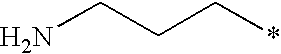 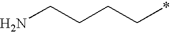 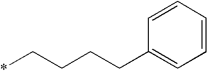 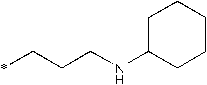 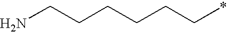 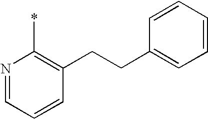 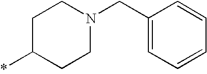 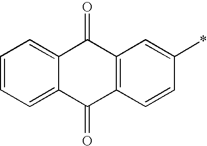 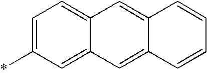 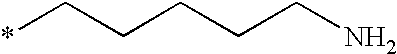 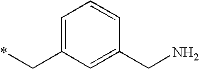 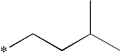 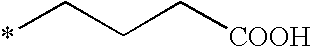 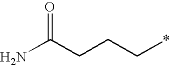 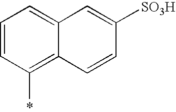 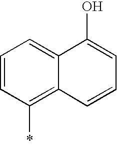 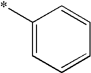 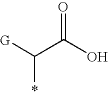
where the asterisk ‘*’ indicates the point of attachment and G represents a side chain of an amino acid.

7. The collection according to claim 1, wherein R³ is selected from the list given in the table below:
R³ 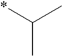 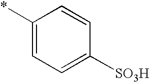 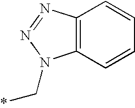 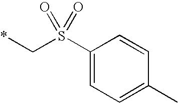 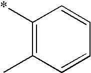 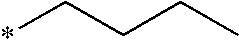
where the asterisk ‘*’ indicates the point of attachment.

8. The collection according to claim 1, wherein R⁴ is selected from the list given in the table below:
R⁴ 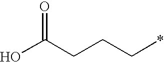 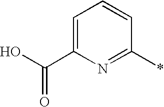 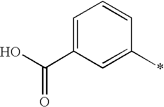 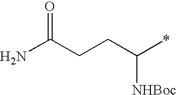 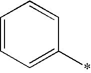 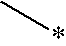 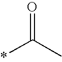 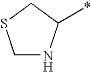 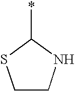 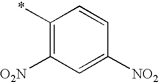 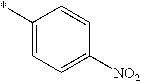 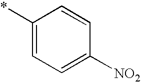 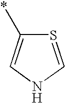 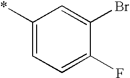 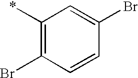 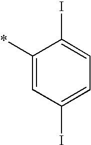 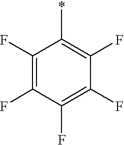 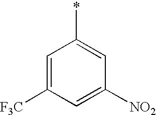 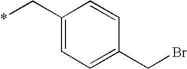 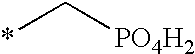 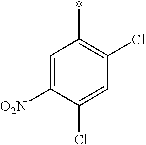 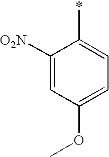 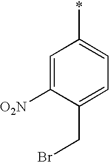 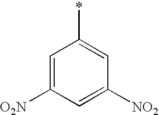 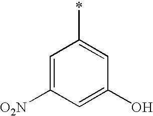 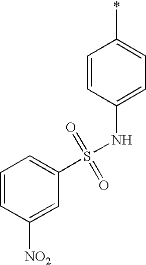 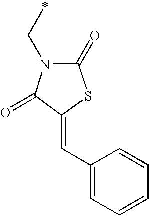
where the asterisk ‘*’ indicates the point of attachment and G represents a side chain of an amino acid.

9. The collection according to claim 1, wherein the others of R^{1a}, R^{1b}, R², R³ and R⁴ for the compounds of formula (I) or the others of R^{1a}, R^{1b}, R³ and R⁴ for the compounds of formula (II) is selected from the group: optionally substituted C₁₋₂₀ alkyl, optionally substituted C₃₋₂₀ heterocyclyl and optionally substituted C₅₋₂₀ aryl.

10. The collection according to claim 1, wherein two or more of the others R^{1a}, R^{1b}, R², R³ and R⁴ for the compounds of formula (I) or two or more of the others of R^{1a}, R^{1b}, R³ and R⁴ for the compounds of formula (II), together with the atoms to which they are bound, form an optionally substituted C₅₋₂₀ heterocyclyl group.

11. The collection according to claim 1, wherein the compound has an analytical label.

12. The collection according to claim 1, wherein the support comprises a glass, gold, a polystyrene, a polysaccharide, a polyacrylamide or a poly(alkoxide).

13. The collection according to claim 1 having at least 10 members.

14. A compound of formula (III) or a compound of formula (IV): 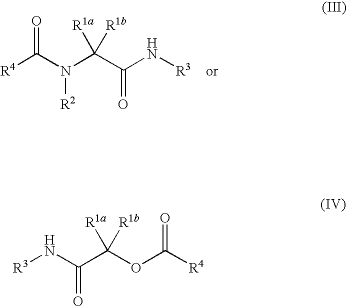
wherein for a compound of formula (III);
one of R^{1a}, R^{1b}, R², R³ and R⁴ is a group comprising a linker attached to a support,
and the others of R^{1a}, R^{1b}, R², R³ and R⁴ are independently selected from optionally substituted C₁₋₂₀ alkyl, optionally substituted C₃₋₂₀ heterocyclyl or optionally substituted C₅₋₂₀ aryl, and R^{1a}, R^{1b} and R² are additionally selected from hydrogen, and R² is additionally further selected from —S(═O)R⁵ and —C(═S)NR⁶R⁷, wherein R⁵, R⁶ and R⁷ are independently optionally substituted C₁₋₂₀ alkyl, optionally substituted C₃₋₂₀ heterocyclyl or optionally substituted C₅₋₂₀ aryl,
or, optionally, two or more of the others of R^{1a}, R^{1b}, R², R³ and R⁴, together with the atoms to which they are bound, may form a ring;
and for a compound of formula (IV);
one of R^{1a}, R^{1b}, R³ and R⁴ is a group comprising a linker attached to a support,
and the others of R^{1a}, R^{1b}, R³ and R⁴ are independently selected optionally substituted C₁₋₂₀ alkyl, optionally substituted C₃₋₂₀ heterocyclyl or optionally substituted C₅₋₂₀ aryl, and R^{1a}, and R^{1b} are additionally selected from hydrogen,
or, optionally, two or more of the others of R^{1a}, R^{1b}, R³ and R⁴, together with the atoms to which they are bound, may form a ring.

15. A separation apparatus for separating a substance from a mixture, wherein the apparatus comprises a compound of formula (III) or a compound of formula (IV) according to claim 14.

16. The separation apparatus according to claim 15 in the form of a chromatographic column.

17. A process for the identification of a immobilised ligand having affinity for a substance, the process comprises the steps of:
obtaining a collection of compounds according to claim 1;
contacting each member of the collection with a mixture comprising a substance; and
analysing the collection to determine to what extent the substance is associated with each collection member.

18. The process according to claim 17, wherein the method further comprises the step of separating the collection from the mixture.

19. A process for the generation of a compound having affinity for a substance, the process comprises the steps of:
obtaining a collection of compounds according to claim 1;
contacting each member of the collection with a mixture comprising a substance;
analysing the collection to determine to what extent the substance is associated with each collection member;
identifying a library member having an affinity for the substance; and
preparing a compound having a structure based on the collection member.

20. The process according to claim 19, wherein the compound having a structure based on the collection member is prepared by
(i) cleaving the linker of a collection member that is determined to be associated with the substance; or
(ii) a method comprising the steps of contacting components A, B, C and D together, wherein
A is R^{1a}COR^{1b};
B is R²—NH₂;
C is R³—NC;
D is R⁴—COOH; and
R^{1a}, R^{1b}, R², R³ and R⁴ are independently selected from optionally substituted C₁₋₂₀ alkyl, optionally substituted C₃₋₂₀ heterocyclyl or optionally substituted C₅₋₂₀ aryl, and R^{1a}, R^{1b} and R² are additionally selected from hydrogen, and R² is additionally further selected from —S(═O)R⁵ and —C(═S)NR⁶R⁷, wherein R⁵, R⁶ and R⁷ are independently optionally substituted C₁₋₂₀ alkyl, optionally substituted C₃₋₂₀ heterocyclyl or optionally substituted C₅₋₂₀ aryl,
or, optionally, two or more of R^{1a}, R^{1b}, R², R³ and R⁴ are connected; or the method comprises the step of contacting components A, C and D together, wherein
A is R^{1a}COR^{1b};
C is R³—NC;
D is R⁴—COOH; and
R^{1a}, R^{1b}, R³ and R⁴ are independently selected optionally substituted C₁₋₂₀ alkyl, optionally substituted C₃₋₂₀ heterocyclyl or optionally substituted C₅₋₂₀ aryl, and R^{1a} and R^{1b} are additionally selected from hydrogen,
or, optionally, two or more of the others of R^{1a}, R^{1b}, R³ and R⁴ are connected.

21. A method for separating a substance from a mixture, the method comprising the steps of:
contacting a mixture comprising a substance with a compound according to claim 14; and
separating the resulting substance-depleted mixture from the substance immobilised to the compound.

22. The method according to claim 21, wherein the method further comprises the step of treating the substance immobilised to the compound with an elutant.

23. A method for determining the presence of a substance in an analytical sample, the method comprising the steps of:
contacting an analytical sample with a compound according to claim 14; and
analysing the compound to determine to what extent the substance is associated with the compound.

24. The method according to claim 23, wherein the compound has an affinity for a substance that is implicated in a particular disease state.

25. The process according to claim 17, wherein the substance is a nucleic acid or a peptide.

26. The process according to claim 25 wherein peptide is a blood protein.

27. The process according to claim 26, wherein the blood protein is a clotting protein.

28. The process according to claim 27, wherein the clotting protein is selected from: Factor VII and Factor VIII, as well as fragments, variants and derivatives thereof.

29. The process according to claim 25 wherein peptide is an immunoglobulin.

30. The process according to claim 29, wherein the immunoglobulin is IgG or fragments, variants and derivatives thereof.

31. A method for the preparation of a collection according to claim 1, the method comprising the step of contacting A, B, C and D together, wherein
A is R^{1a}COR^{1b};
B is R²—NH₂;
C is R³—NC;
D is R⁴—COOH; and
one of R^{1a}, R^{1b}, R², R³ and R⁴ is a group comprising a linker attached to a support,
and the others R^{1a}, R^{1b}, R², R³ and R⁴ are independently selected from optionally substituted C₁₋₂₀ alkyl, optionally substituted C₃₋₂₀ heterocyclyl or optionally substituted C₅₋₂₀ aryl, and R^{1a}, R^{1b} and R² are additionally selected from hydrogen, and R² is additionally further selected from —S(═O)R⁵ and —C(═S)NR⁶R⁷, wherein R⁵, R⁶ and R⁷ are independently optionally substituted C₁₋₂₀ alkyl, optionally substituted C₃₋₂₀ heterocyclyl or optionally substituted C₅₋₂₀ aryl,
or, optionally, two or more of the others of R^{1a}, R^{1b}, R², R³ and R⁴ are connected, wherein the step is repeated one or more times, and for each repeat, one or more of A, B, C or D is varied;
or the method comprises the step of contacting components A, C and D together, wherein
A is R^{1a}COR^{1b};
C is R³—NC;
D is R⁴—COOH; and
one of R^{1a}, R^{1b}, R³ and R⁴ is a group comprising a linker attached to a support,
and the others of R^{1a}, R^{1b}, R³ and R⁴ are independently selected optionally substituted C₁₋₂₀ alkyl, optionally substituted C₃₋₂₀ heterocyclyl or optionally substituted C₅₋₂₀ aryl, and R^{1a} and R^{1b} are additionally selected from hydrogen,
or, optionally, two or more of the others of R^{1a}, R^{1b}, R³ and R⁴ are connected,
wherein the step is repeated one or more times, and for each repeat, one or more of A, C or D is varied.

32. A method for the preparation of a compound according to claim 14, the method comprises the step of contacting components A, B, C and D together, wherein
A is R^{1a}COR^{1b};
B is R²—NH₂;
C is R³—NC;
D is R⁴—COOH; and
one of R^{1a}, R^{1b}, R², R³ and R⁴ is a group comprising a linker attached to a support,
and the others R^{1a}, R^{1b}, R², R³ and R⁴ are independently selected from optionally substituted C₁₋₂₀ alkyl, optionally substituted C₃₋₂₀ heterocyclyl or optionally substituted C₅₋₂₀ aryl, and R^{1a}, R^{1b} and R² are additionally selected from hydrogen, and R² is additionally further selected from —S(═O)R⁵ and —C(═S)NR⁶R⁷, wherein R⁵, R⁶ and R⁷ are independently optionally substituted C₁₋₂₀ alkyl, optionally substituted C₃₋₂₀ heterocyclyl or optionally substituted C₅₋₂₀ aryl,
or, optionally, two or more of the others of R^{1a}, R^{1b}, R², R³ and R⁴ are connected; or the method comprises the step of contacting components A, C and D together, wherein
A is R^{1a}COR^{1b});
C is R³—NC;
D is R⁴—COOH; and
one of R^{1a}, R^{1b}, R³ and R⁴ is a group comprising a linker attached to a support,
and the others of R^{1a}, R^{1b}, R³ and R⁴ are independently selected optionally substituted C₁₋₂₀ alkyl, optionally substituted C₃₋₂₀ heterocyclyl or optionally substituted C₅₋₂₀ aryl, and R^{1a} and R^{1b} are additionally selected from hydrogen,
or, optionally, two or more of the others of R^{1a}, R^{1b}, R³ and R⁴ are connected.

33. A collection according to claim 1 obtained by the method according to claim 31.
